(19) United States
(12) Patent Application Publication
(10) Pub. No.: US 2004/0014652 A1
(43) Pub. Date: Jan. 22, 2004

(54) Tumor activated prodrug compounds and methods of making and using the same

(76) Inventors: Andre Trouet, Herent (BE); Vincent Dubois, Fleurus (BE); Arnold Oronsky, Los altos Hills, CA (US)

Correspondence Address: Elie H Gendloff, Amster Rothstein & Ebenstein, 90 Park Avenue, New York, NY, 10016, US

(21) Appl. No.: 10/296,954

(22) PCT Filed: May 29, 2001
(86) PCT No.: PCT/EP01/06106

(30) Foreign Application Priority Data
Jun. 1, 2000 (EP) 00870130.2
Dec. 18, 2000 (EP) 00870306.8

(51) Int. Cl. A61K 38/17; C07K 14/00

(52) U.S. Cl. 514/12; 530/324; 530/350

(57) ABSTRACT

The invention is directed to novel prodrug compounds, compositions comprising the prodrug compounds, methods of making the prodrug compounds and methods of using the prodrug compounds. The prodrug compounds comprise a biologically active entity linked to a masking moiety via a linking moiety. The prodrug compounds are selectively activated at or near target cells and display lower toxicity and possibly a longer in vivo or serum half-life than the corresponding naked biologically active entity.

### 1. FIELD OF THE INVENTION

The present invention relates to novel prodrug compounds, to pharmaceutical compositions comprising the novel prodrug compounds and to methods of using the compounds to inhibit the growth of tumors and/or to treat malignant tumors and/or tumorigenic cancers.

### 2. BACKGROUND

Cancer is currently the second largest killer in the developed world with more than 6 million deaths per year, a figure that is expected to double by 2022. Despite efforts to improve the efficacy of treatments, relatively low cure rates have been achieved to date.

Current attempts at cancer therapy suffer from several major deficiencies. First, most available cancer therapies consist of drugs that act on rapidly dividing cells. However, most cancers are diagnosed at a time when the proportion of rapidly dividing tumor cells is reduced. Second, normal tissues that contain rapidly dividing cell populations are also affected by the current anticancer entities. The resulting toxicity forces reduced dosage levels and reduced frequencies of treatment. Third, tumor cells are genetically unstable and have high mutation rates. As a result, tumors frequently develop resistance to treatment. Finally, current anticancer therapeutics, like most therapeutic entities, can be unstable to the enzymes and other degradation proteins in the blood and serum.

New cytotoxic drugs are regularly entering the clinic but their use remains hampered by their toxic side effects, the high rate of induced resistance and, in some instances, their poor blood stability. In particular, extracellularly active entities and polypeptides have recently been identified that have cytotoxic or cytostatic effects on tumor cells in vitro and/or in vivo. For instance, TNFα, a cytokine with diverse effects on cells both in vitro and in vivo, exhibits cytotoxic and cytostatic effects on tumor cell types in vitro. However, the growth of many normal cell types, such as endothelial cells, smooth muscle cells, adipocytes, fibroblasts and keratinocytes is also Inhibited by TNFα in vitro. Sensitivity of human endothelial cells to TNFα correlates with their rate of proliferation. Multiple clinical studies (phase I and II) have been carried out with recombinant TNFα as an anticancer entity without major therapeutic effect. TNFα-induced systemic toxicity and acquired resistance to TNFα ar two major roadblocks towards the success of TNFα as an antineoplastic entity.

Thus, a need remains for the availability of effective antineoplastic entities for use in treating solid tumor types. Especially needed are antineoplastic entities that exhibit some degree of selectivity and/or specificity for tumor cells or that can be delivered at dosages that achieve therapeutic benefit with little or no toxicity.

### 3. SUMMARY OF THE INVENTION

In one aspect, the present invention provides novel prodrug compounds that have improved therapeutic properties. In general, the novel prodrugs comprise a biologically active entity linked to a masking moiety via a linking moiety. By virtue of the nature of the masking and linking moieties, when included in the prodrugs of the present invention, the biologically active entities can be administered to tumor cells and to endothelial cells involved in tumor neoangiogenesis in a selective manner. Moreover, the prodrugs of the invention will typically exhibit lower toxicity and possibly a longer in vivo or serum half-life than the corresponding naked biologically active entity.

The invention is based, in part, on the discovery that peptides having certain amino acid sequences are specifically cleaved by proteases and/or peptidases in the extracellular medium at or near tumor cells and certain endothelial cells involved in neoangiogenesis (“target cells”). Particularly, peptides that may be specifically cleaved in the target extracellular milieu include those having the amino acid sequence (Leu)_{y}Ala-Leu)ₓAla-Leu and (Leu)_{y}(Ala-Leu)ₓAla-Phe, where y is 0 or 1 and x is 1, 2 or 3. Utilizing these peptides as components of linking moieties to link the biologically active entities and masking moieties of the prodrugs of the invention together permits the biologically active entity to be selectively released or liberated in vivo at or near a tumor or target cell.

The peptidase that cleaves the linking moiety is not unique to tumors or tumor cells. Healthy cells also produce peptidases capable of cleaving the linking moiety. However, it has been discovered that significantly more cleavage is observed around tumor cells. While not intending to be bound by any particular theory of operation, it is believed that tumor cells excrete significantly higher concentrations of the cleaving peptidases than do healthy cells, which accounts for the observed higher cleavage in their vicinity. Thus, while the peptidases are not unique to tumors or tumor cells, the prodrugs of the present invention exploit the observed differences in cleavage surrounding tumor cells and healthy cells to selectively deliver biologically active entities to tumor cells. Moreover, such peptidases are also released by endothelial cells involved in tumor angiogenesis at higher concentrations than healthy cells, permitting the preparation and selective delivery of antiangiogenic compounds to these endothelial cells. Thus, by virtue of the linking moiety and the observed differences in the amount of specifically cleaving peptidases produced and/or secreted by the target and healthy cells, the prodrugs of the invention permit compounds that are cytotoxic or cytostatic to be selectively delivered to the target cells, thereby providing a selective and safe means of delivering the cytotoxic and/or cytostatic agents to patients to treat tumorigenic conditions, such as malignant tumorigenic cancers.

The masking moiety of the prodrugs is linked to the biologically active entity via the linking moiety. The masking moiety, acting alone or together with the linking moiety, blocks or inhibits the biological activity of the biologically active entity. Moreover, the masking moiety prevents non-specific degradation and/or cleavage of the linking moiety by, for instance, peptidases present in serum, and hence prevents non-selective release of the biologically active entity. While not intending to be bound by any theory of operation, it is believed that the biological activity of the biologically active entity may be blocked or inhibited by several mechanisms, such as, for example, steric hindrance caused by the masking moiety-linking moiety assembly and/or masking of charged groups on the biologically active entity that are required for biological activity. Masking moieties suitable to prevent in vivo non-specific degradation of the linking moiety can range from chemical modification of the exposed terminus of the linking moiety, such as an amino or carboxy terminal modification, to small molecules such as drugs and genetically non-encoded amino acids or other enzymatically non-degradable amino acids (including, e.g., D-amino acids, β-amino acids, γ-amino acids and the like) to large molecules such as polypeptides or other biological or non-biological polymers. The masking moiety may be biologically inert, or it may itself have biological activity, such as any of the biologically active entities described infra or any other drug, even a drug with no cytotoxic or cytostatic activity.

The biologically active entity is typically a molecule or construct that is cytotoxic and/or cytostatic to cells, but may be any agent or drug useful in the diagnosis and/or treatment of tumors and/or tumorigenic cancers. The types of molecules suitable for use as biologically active entities in the prodrugs of the invention can vary widely. For formulation as a prodrug, the biologically active entity should comprise, or be modified to comprise, a reactive group capable of forming a covalent linkage with the appropriate terminus of the linking moiety. If the biologically active entity must be modified to effect linkage, it should be modified in a manner such that when it is selectively released it retains substantial biological activity. Thus, the biologically active entities can range from small organic compounds such as anthracyclines, doxorublcin, daunorubicin, folic acid derivatives, vinca alkaloids, calicheamycin, mitoxantrone, cytosine arabinosides, adenosine arabinosides, fludarabine phosphate, melphalan, bleomycin, mitomycin, L-canavanine, taxoids, camptothecin, proteasome inhibitor, farnesyl transferase inhibitor, epothilone, discodermolide, maytansinoids, platinum derivatives, duocarmycins, combrestastatin and epipodophyllotoxins, to biological oligomers such as oligonucleotides, oligopeptides and oligosaccharides, to large biological polymers such as nucleic acids and polypeptides.

Those of skill in the art will recognize that certain biologically active entities, such as polypeptides, may contain a plurality of reactive groups that are capable of forming a covalent linkage with a terminus of the linking moiety (for example, polypeptides having a plurality of amino acids with primary amine or carboxyl side chains, such as arginine, lysine, aspartate, and glutamate). Any or all of the available reactive groups may be linked to linking moiety. The various linking moieties may be the same or different, and each linking moiety is in turn linked to a masking moiety, which may be the same or different for each linking moiety. Thus, depending on the number of reactive groups available on the biologically active entity, the prodrugs of the invention may comprise one or a plurality of the same or different linking moieties.

The biologically active entity may act intracellularly or extracellularly to exert its biological effect. For example, the biologically active entity may be a small organic compound that is capable of traversing cell membranes and exerting its cytotoxic and/or cytostatic effects inside the cell (e.g., anthracyclines). Alternatively, the biologically active entity may be a molecule which binds an extracellular domain of a receptor and triggers cell death (e.g., cytokines such as TNFα).

The biologically active entity may also be an entity that acts intracellularly but that is either incapable of traversing the cell membrane or does not efficiently traverse the membrane on its own. Such intracellularly active biologically active agents may be coupled to a peptide that facilitates transport into the cell or cell nucleus. Selective delivery of a biologically active entity directly into the nucleus of a target cell may improve the selectivity of the biologically active entity and may overcome drug resistance to the biologically active entity. Intracellularly active agents that are not capable of traversing the cell membrane Include intracellularly active polypeptides such as granzyme B, many antisense DNAs and RNAs, many ribozymes and genes useful for gene therapy. In addition, while many cytostatic or cytotoxic small molecules traverse the membrane on their own, formulation of such small molecules, for example doxorubicin and daunorubicin, with a transport peptide may enhance their membrane permeation properties. In this aspect of the invention, the masking moiety, alone or in combination with the linking moiety, prevents the biologically active entity—transport peptide construct from entering cells prior to selective cleavage of the linking moiety. Prodrugs of the invention in which the biologically active entity includes a transport peptide permit selective delivery of biologically active entities to tumors and/or target cells that otherwise would be unable to selectively traverse the cell membrane or would do so with a low efficiency.

In one preferred embodiment of the invention, the biologically active entity is an extracellularly active cytotoxic or cytostatic entity—i.e., an entity that inhibits the growth of and/or kills a cell without having to enter the cell. The extracellularly active cytotoxic or cytostatic entity may be virtually any molecule that can be linked to a masking moiety. Non-limiting examples include, e.g., peptides, lytic peptides, anti-angiogenic peptides and polypeptides. Preferred polypeptides are those that have a cytotoxic or cytostatic effect on tumor cells and include, but are not limited to, TNFα, IFN-α, IFN-γ, IL-1, IL-2, IL-6, IGF-1 antagonists, thrombospondin-1 derived peptides, substance P antagonists, TRAIL (Apo-2 ligand) and Fas ligand. Prodrugs of the invention including such extracellularly active biologically active entities permit selective delivery of the entities to target cells, thereby reducing their toxicity to the patient.

As discussed above, the masking moiety may also have biological activity. Thus in another embodiment, the prodrugs of the invention are dual prodrugs—i.e., prodrugs in which both the masking moieties and biologically active moieties exert biological effect. In one embodiment, the dual prodrugs comprise two intracellularly active entities. The dual prodrugs include prodrugs that selectively deliver two biologically active small molecules to target cells. Other dual prodrugs include prodrugs that deliver two molecules that act in concert against a target cell. For instance, dual prodrugs can deliver a polypeptide that interacts with an extracellular receptor and a small molecule that acts on an intracellular target. Alternatively, the dual prodrugs may comprise two drugs that are often prescribed in combination such as a cytotoxic agent and an antibiotic, or two drugs that act synergistically such as TNFα and doxorubicin or other combinations of biologically active compounds as are well known in the art. Preferred dual prodrugs are those that deliver two entities that act in concert against a target cell.

When the biologically active entity is a small organic intracellularly active compound capable of traversing the cell membrane, it is either included In the prodrug compositions of the Invention in association with a transport peptide that acts to enhance its membrane permeation properties or as a dual prodrug in which the masking moiety also has some biological activity. Other biologically active entities, such as the extracellularly active entities and other entities described supra may be included in the prodrugs of the invention with biologically inert masking moieties or as dual prodrugs in which the masking moiety also has some biological activity.

In another aspect, the present invention provides pharmaceutical compositions comprising the prodrugs of the present invention. The pharmaceutical compositions generally comprise one or more prodrugs of the invention and a pharmaceutically acceptable carrier, excipient or diluent. Preferably, the pharmaceutical compositions comprise an amount of prodrug that provides therapeutic benefit.

In another aspect, the present invention provides methods of inhibiting the growth or proliferation of a tumor or a tumor cell or an endothelial cell involved in tumor neoangiogenesis. The method comprises contacting a tumor or a target cell with an amount of a prodrug or pharmaceutical composition of the invention effective to inhibit the growth or proliferation of the tumor cell or target cell. The method can be practiced to inhibit the growth or proliferation of tumors and/or target cells in vivo, in vitro or ex vivo.

In a final aspect, the present invention provides methods of treating solid tumors and their metastases and tumorigenic cancers. The method generally comprises administering to an animal, including a human, having a solid tumor and/or cancer an amount of a prodrug or pharmaceutical composition of the invention effective to halt the progression of tumor growth, thereby treating the animal. Preferably, an amount of prodrug or pharmaceutical composition effective to shrink or eradicate the tumor is administered.

### 4. BRIEF DESCRIPTION OF THE FIGURES

FIG. 1A illustrates an exemplary prodrug of the present invention;

FIG. 1B illustrates a prodrug of FIG. 1A near a healthy cell;

FIG. 1C illustrates the activation of the prodrug of FIG. 1A when near a tumor cell;

FIG. 2 illustrates the in vivo function of a prodrug of the present invention;

FIG. 3A illustrates the structure of two linking moieties of the present invention;

FIG. 3B illustrates the specific cleavage of a prodrug of one polarity in the extracellular milieu at or near a tumor or target cell;

FIG. 3C Illustrates the specific cleavage of a prodrug of a second polarity in the extracellular milieu at or near a tumor or target cell

FIG. 3D Illustrates th specific cleavage of a prodrug comprising a spacing moiety;

FIG. 3E Illustrates the specific cleavage of a dual polarity prodrug;

FIG. 4A Illustrates a general scheme for the preparation of a prodrug of the present invention using protecting moieties;

FIG. 4B illustrates the preparation of a prodrug of the present invention;

FIG. 4C illustrates the preparation a prodrug comprising a spacing moiety; and

FIG. 4D illustrates the preparation of a dual polarity prodrug

### 5. DETAILED DESCRIPTION OF THE PREFERRED EMBODIMENTS

5.1 Abbreviations.

As used herein, the abbreviations for the genetically encoded amino acids are conventional and are as follows:

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

To avoid confusion with the various formulae used herein, genetically-encoded amino acid residues are generally designated with the three-letter abbreviations. Unless otherwise noted, the three-letter abbreviations designate L-enantiomers of the genetically encoded amino acids. Amino acids in the D-configuration will be explicitly labeled. For xample, “Arg” designates L-arginin and “D-Arg” designates D-arginine. When one-letter abbreviations are used, upper case letters designate amino acids in the L-configuration and lower case letters designate amino acids in the D-configuration. For example, “R” designates L-arginine and “r” designates D-arginine. When a peptide or polypeptide sequence is represented as a series of one-letter or three-letter abbreviations, unless specifically noted otherwise, it will be understood that the left-hand direction is the amino terminal direction and the right-hand direction is the carboxy terminal direction, in accordance with standard usage and convention.

5.2 Definitions

As used herein, the following terms shall have the following meanings:

“Biologically active entity” refers to a molecule or construct that exerts a biological effect on a target cell, as defined herein. Typically, the entity is cytotoxic and/or cytostatic toward target cells or sensitizes target cells to the action of another cytotoxic or cytostatic entity.

“Linking moiety” refers to a molecular moiety with a structure as described herein that links a biologically active entity to a masking moiety and that is susceptible to specific, selective cleavage at or near a tumor or a target cell, as defined herein.

“Normal cells”, “normal tissues”, “healthy cells” and “healthy tissues” refer to cells and/or tissues that are not involved in tumor formation, growth and metastasis. As used herein, endothelial cells involved in tumor neoangiogenesis are not included in the definition of normal or healthy cells.

“Masking moiety” refers to a molecular moiety that, when linked to a biologically active entity via a linking moiety, is capable, together with the linking moiety, of masking the biological activity of the entity and is capable of preventing the non-specific degradation of the linking moiety.

“Polypeptide” refers to a polymer of two or more amino acids. The term also includes mimics of polymers of amino acids known to those of skill in the art (e.g. peptidomimetics) and derivatives of polymers of amino acids (e.g., glycopeptides). The term “peptide” refers to a polypeptide having from about 2 to about 40 to 50 amino acids.

“Specific cleavage” refers to cleavage that is sequence dependent. Thus, specific cleavage of a linking moiety is cleavage that occurs as the result of a peptidase recognizing and cleaving the particular amino acid sequence of the linking moiety.

“Specific cleavage” is therefore a property of the amino acid sequence of the linking moiety and is to be distinguished from cleavage and/or degradation caused by non-specific means, such as non-specific degradation by exopeptidases present in the serum or gut.

“Selective cleavage” refers to the enhanced or preferential specific cleavage achieved at or near a target cell. Thus, while the specific cleavage of the linking moiety is not unique to target cells, the greater cleavage achieved at or near target cells renders the cleavage “selective” for purposes of the present invention.

“Target cell” refers to a tumor cell or to an endothelial cell involved in tumor neoangiogenesis.

5.3 The Invention

The present invention provides novel prodrugs comprising a biologically active entity linked to a masking moiety via a linking moiety. The prodrugs of the present invention are generally compounds according to formula (I):

(M−L¹)ₙ−B (I)

or a pharmaceutically acceptable salt thereof, wherein M is a masking moiety, L¹ is a linking moiety, B is a biologically active entity and n is a positive integer from 1 up to the total number of reactive groups in the biologically active entity. The masking moiety prevents the non-specific degradation of the linking moiety and (M−L¹)ₙ masks the biological activity of the biologically active entity, rendering it inactive until specifically released. The linking moiety is susceptible to specific, selective cleavage at or near a tumor or target cell. By virtue of the specific and selective cleavage of the linking moiety in vivo, an entity formulated as a prodrug according to the present invention displays improved selectivity relative to the naked entity. The entity formulated as a prodrug may also display improved stability relative to the naked entity because the masking moiety prevents degradation of the prodrug in blood or serum.

5.4 Linking Moiety

The linking moiety can comprise any molecule that is susceptible to specific cleavage at or near a tumor or a target cell. The linking moiety is covalently linked to the masking moiety and to the biologically active entity, thereby linking the two together.

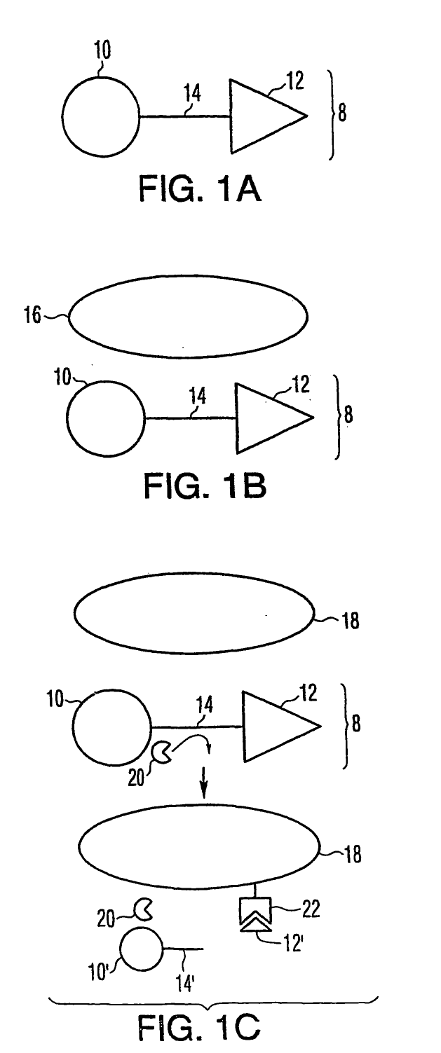

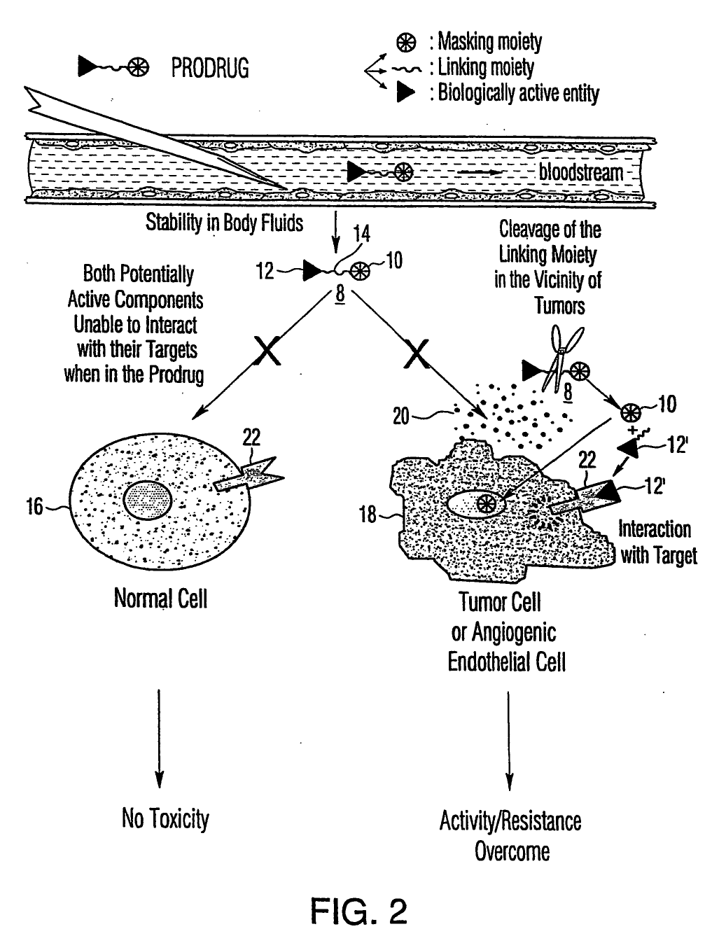

While not intending to be bound by theory of operation, it is believed that tumors and target cells secrete into the extracellular medium a factor or factors such as proteases or peptidases that are capable of specifically cleaving the linking moiety. It Is also believed that endothelial cells involved In tumor neoangiogenesis display the same secretory activity. While healthy cells also produce a factor or factors that specifically cleave the linking moiety, tumor cells and endothelial cells involved in tumor neoangiogenesis excrete a significantly higher concentration of the factor thereby permitting specific and selective cleavage at or near tumors and/or target cells. The resulting improved selectivity of action of the biologically active entity is illustrated in FIG. 1. FIG. 1A illustrates an exemplary prodrug 8 of the present invention comprising a masking moiety 10 linked to a biologically active entity 12 via a linking moiety 14. In FIG. 1B, prodrug 8 is not active near healthy cell 16 because masking moiety 10, alone or in combination with linking moiety 14, masks the biological activity of biologically active entity 12 and linking moiety 14 is relatively stable at or near healthy cell 16. In FIG. 1C, target (tumor or angiogenic endothelial) cell 18 secretes a factor 20 which is capable of specifically cleaving the linkage between linking moiety 14, liberating released biologically active entity 12′. Liberated biologically active entity 12′ is free to exert its activity on target cell 18. For instance, biologically active entity 12′ might bind receptor 22 on the surface of target cell 18, thereby initiating an intracellular cascade leading to the apoptosis or another form of death of tumor cell 18. Because normal cell 16 does not secrete factor 20, or secretes very little factor 20, prodrug 8 remains intact near normal cell 16. Thus, biologically active entity 12 is not active near normal cell 16. As illustrated in FIG. 2, formulation of the biologically active entity as a prodrug can reduce the toxicity of the biologically active entity.

Factor 20 can be any molecule or condition in the environment at or near target cell 18 that is capable of specifically cleaving linking moiety 14. While not intending to be bound by theory, it is believed that factor 20 is a protease or peptidase selectively secreted by target cells. However, since all proteases or peptidases that specifically cleave the linking moiety have not yet been identified, factor 20 can be any condition that is capable of specifically cleaving the linking moiety. For instance, factor 20 might even be low pH conditions near a target cell. Factor 20 is selectively present at or near target cells. It can be present at or near target cells exclusively, or it can be enriched at or near target cells such that prodrug 8 is cleaved preferentially at or near target cells and administration of prodrug 8 displays improved selectivity for target cells relative to administration of the naked biologically active entity 12 or released biologically active entity 12′.

Preferred linking moieties are peptides that are susceptible to specific cleavage at or near target cells. For example, it has been discovered that peptides having the amino acid sequence (Leu)_{y}(Ala-Leu)ₓAla-Leu and p ptides having the amino acid sequence (Leu)_{y}(Ala-Leu)ₓAla-Phe (wherein y=0 or 1 and x=1, 2, or 3) are specifically cleaved by a factor in the extracellular milieu near target cells. Preferred peptide linking moieties comprise the amino acid sequence Ala-Leu-Ala-Leu (SEQ ID NO:1), Leu-Ala-Leu-Ala-Leu (SEQ ID NO:2), Leu-Ala-Leu (SEQ ID NO:3), Leu-Ala (SEQ ID NO:4) or Leu-Ala-Phe (SEQ ID NO:5).

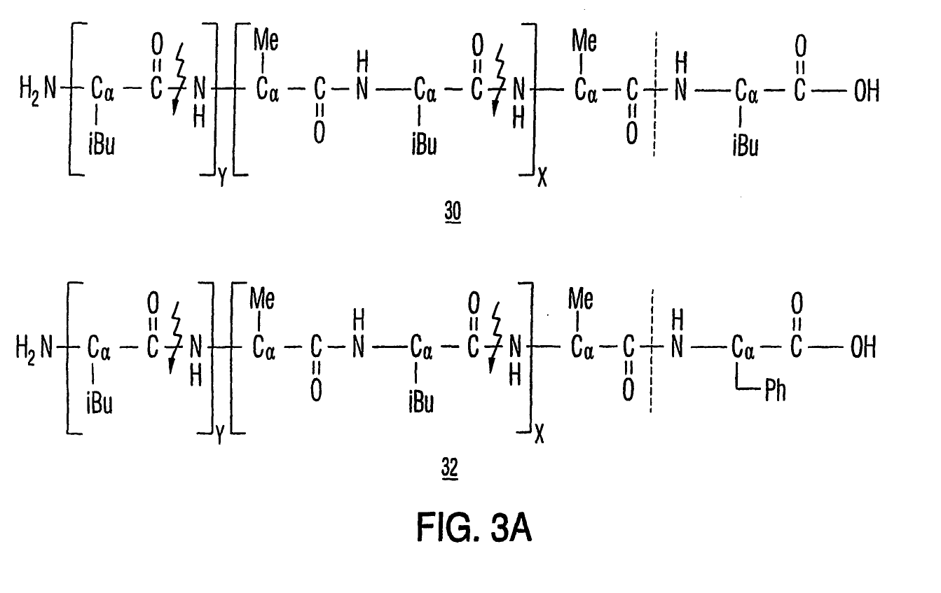

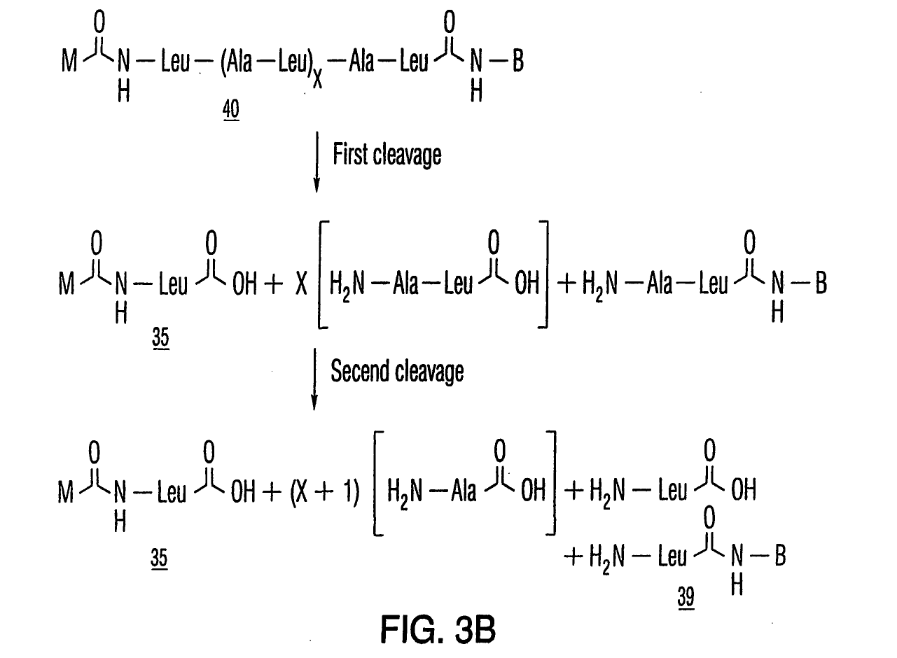

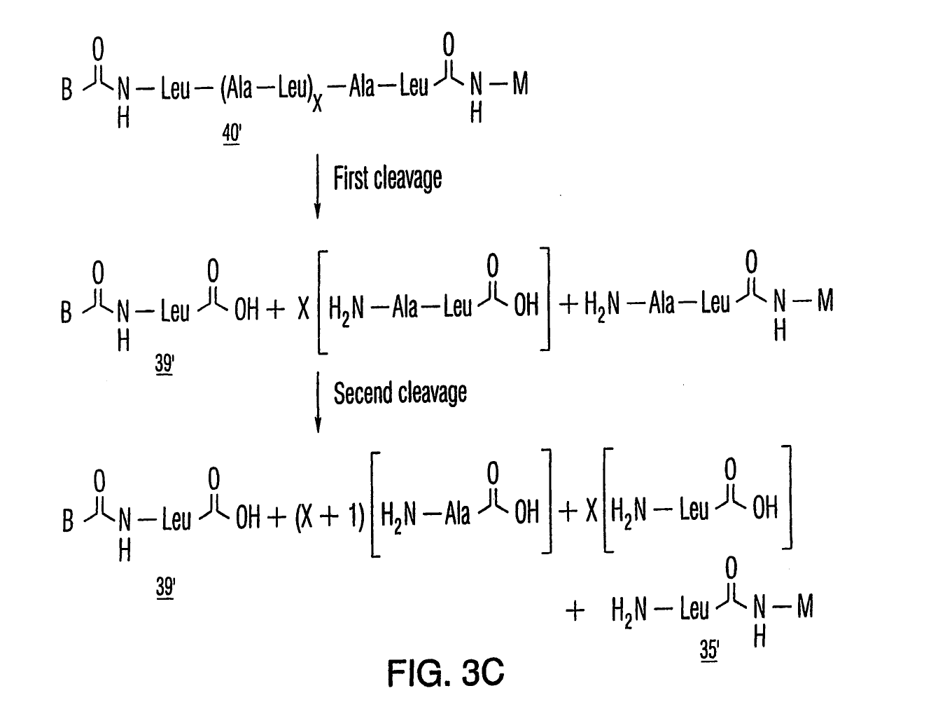

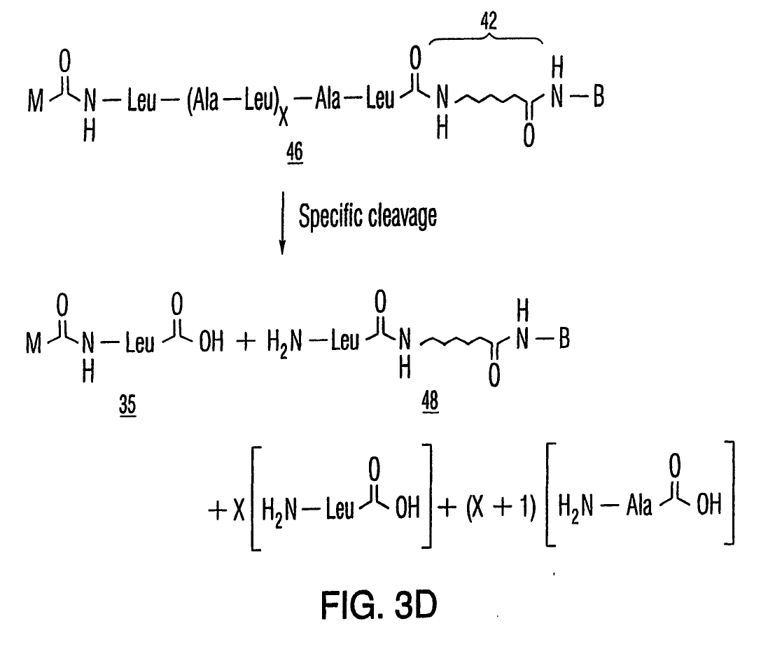

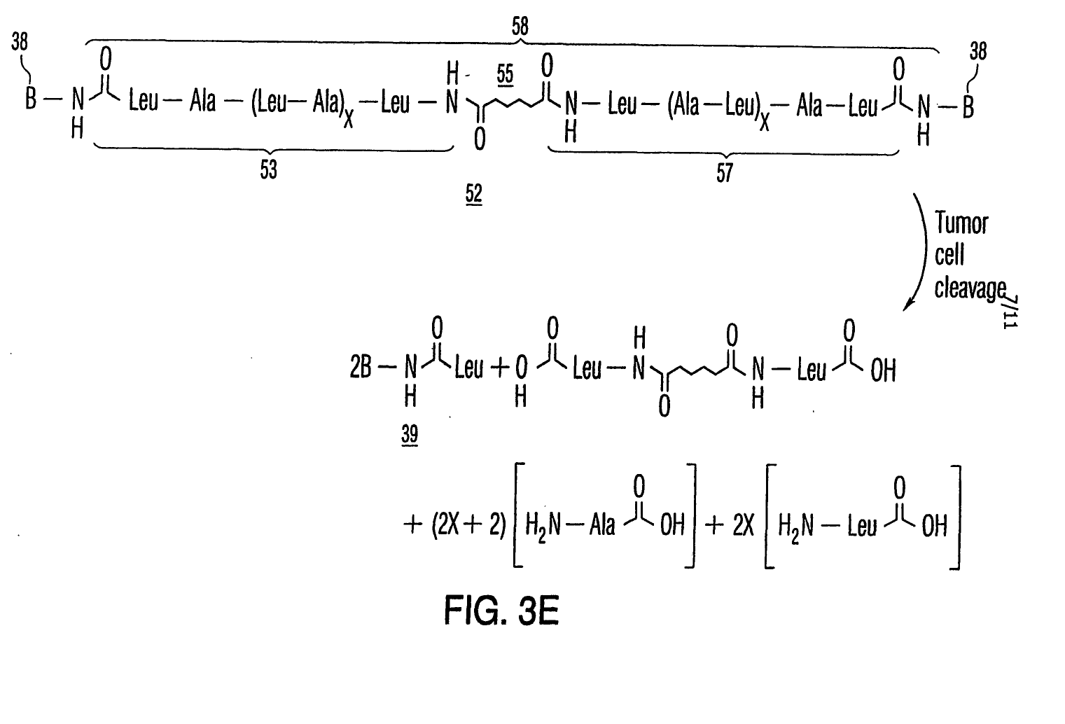

While not intending to be bound by any particular theory of operation, it is believed that the linking moiety is specifically cleaved at two sites. A first protease or peptidase is believed to cleave the first amide bond of each Leu-Ala-Leu sequence of the linking moiety (i.e. the amide bond between the N-terminal Leu and the Ala in the sequence), as illustrated by solid arrows in FIG. 3A. In FIG. 3A, iBu represents isobutyl; Me represents methyl; Ph represent phenyl and x and y are as previously defined above. A second protease or peptidase is believed to subsequently cleave the amide bond of the Ala-Leu or Ala-Phe residues of the linking moiety, as indicated by dashed lines in FIG. 3A. However, specific cleavage may also occur at other amide bonds or at other bonds within the linking moiety.

The linking moiety is covalently linked to the biologically active entity via one terminus and to the masking moiety via the other terminus. The “polarity” of the linkages, Le., whether the N-terminus of the linking moiety is linked to the biologically active entity or to the masking moiety, may or may not be critical, and will depend upon the identities of the biologically active entity and masking moiety.

The different possible polarities of the linkage and the resultant specific cleavage products are illustrated in FIG. 3B and FIG. 3C with linking moiety 30. Referring to FIG. 3B, in prodrug 40, masking moiety M is linked to the amino terminus of linking moiety 30 and biologically active entity B is linked to the carboxy terminus of linking moiety 30. As illustrated, both linkages are amide linkages, although other stable linkages could also be used, as will be described in more detail in a later section. The first protease or peptidase is believed to cleave the amide bonds between the first Leu and the Ala of the Leu-Ala-Leu sequences of the linking moiety thereby liberating released masking moiety 35. A second protease or peptidase cleaves the N-terminal Ala residue from the amino terminus of the derivative of the biologically active entity thereby liberating released biologically active entity 39. Thus, when the linking moiety is a peptide with the amino acid sequence of Leu-Ala-Leu-Ala-Leu, cleavage of the linking moiety is believed to liberate a Leu derivative 35 of the masking moiety and a Leu d rivative of the biologically active entity 39. The Leu derivative of the masking moiety 35 has a free carboxy terminus, and the Leu derivative of the biologically active entity has a fre amino terminus.

Referring to FIG. 3C, if the polarity of the linkage is reversed, such that the biologically active entity is attached to the amino terminus and the masking moiety is attached to the carboxy terminus (prodrug 40′), specific cleavage yields released biologically active entity 39′ and released masking moiety 35′. Thus, when the linking moiety is a peptide with the amino acid sequence of Leu-Ala-Leu-Ala-Leu, cleavage of the linking moiety is believed to liberate a Leu derivative 35 of the masking moiety and a Leu derivative of the biologically active entity 39. The Leu derivative of the masking moiety 35 has a free amino terminus, and the Leu derivative of the biologically active entity has a free carboxy terminus.

Those of skill in the art will recognize that, while in most instances the masking moiety and the biologically active entity will be linked directly to the termini of the linking moiety, in some instances it may be desirable to space the linking moiety away from either or both the masking moiety and biologically active entity with a spacing moiety. An example of the use of a spacing moiety between the linking moiety and the biologically active entity is illustrated in FIG. 3D.

In FIG. 3D, a prodrug 46 according to the invention including a spacing moiety (illustrated in this example as 6-aminocaproic acid) is specifically cleaved to yield released masking moiety 35 and released biologically active entity 48. Again, while amide linkages are illustrated, other stable linkages could be used, as will be described in more detail in a later section.

The spacing moiety can be long or short, rigid, semi-rigid or flexible, hydrophobic or hydrophilic, depending upon the particular application. Thus, the spacing moiety may be any molecule having reactive groups capable of forming covalent linkages with the linking moiety and the biologically active entity and/or masking moiety. Reactive groups that are capable of forming suitable linkages with the amino and carboxy termini of linking moieties are described in more detail below in connection with the biologically active entity and masking moiety. Molecules suitable for use as spacing moieties include, but are not limited to, peptides such as polyglycine (flexible) or polyproline (rigid), aminoalkylcarboxylic acids (e.g., 4-aminobutanoic acid, 5-aminopentanoic acid, 6-aminocaproic acid, etc.), polyalkylene oxides such as polyethylene glycol and others as will be apparent to those of skill in the art. If used, the spacing moiety should preferably be a molecule which is biologically inert i.e., a molecule that does not elicit an immunological response or other adverse or toxic response and should not significantly adversely affect the biological activity of released biologically active entity 48.

In certain embodiments of the invention where it is desirable to link two biologically active entities together, such as the dual prodrugs described infra, the spacing moiety may be used to link two peptide sequences that are specifically cleavable, as illustrated in FIG. 3E. Referring to FIG. 3E, dual prodrug 52 comprises two biologically active entities 38 linked together via dual polarity linking moiety 58. Dual polarity linking moiety 58 comprises two specifically cleavable peptides 53 and 57, which themselves may constitute linking moieties as discussed above, linked together via spacer moiety 55. Spacer 55 is a dicarboxylic acid that is linked to the amino termini of both peptides 53 and 57 such that prior to attachment to biologically active entities 38, linking moiety 58 has two carboxy termini. Specific cleavage of dual prodrug 52 yields released biologically active entities 39 and short peptide derivatives of linking moiety 58. Optional spacing moieties could be included between the linking moiety 58 and one or both biologically active entities 39, as previously described. These dual polarity linking moieties are described in more detail below in connection with the dual prodrugs of the invention.

In embodiments of Formula (I), where n is greater than 1, all the linking moieties of the prodrug may be the same, some of the linking moieties may be the same and others different, or each linking moiety may be different. Similarly, all or some of the masking moieties (discussed in detail below) can be the same, or they can be different. In addition, one, some, all or none of the linking moieties may include spacing moieties and when included, the various spacing moieties may be the same or different.

5.5 Masking Moiety

At a minimum, the masking moiety prevents the non-specific cleavage and/or degradation of the linking moiety and, alone or together with said linking moiety, inhibits the biological effects of the biologically active entity. It may also, as will be discussed in more detail below, provide the prodrug composition with additional favorable properties such as longer in vivo half-life, increased stability, higher solubility, etc. Preferred masking moieties are those that are stable in vivo, nontoxic to healthy cells and non-immunogenic.

In its simplest form, the masking moiety may comprise a chemical modification of the exposed terminus of the linking moiety that prevents the linking moiety from being non-specifically cleaved or degraded in vivo, such as, for example by non-specific exopeptidases that may non-specifically degrade the linking moiety in vivo and prematurely release the biologically active entity. For example, when the exposed terminus of the linking moiety is the amino terminus, amino terminal acetylation of the linking moiety may provide sufficient resistance to degradation to serve as a masking moiety. When the exposed terminus of the linking moiety is the carboxy terminus, carboxy terminal modification, such as amidation or esterification, of the linking moiety may similarly provide sufficient resistance to degradation. Other chemical modifications that inhibit or prevent non-specific degradation and/or cleavage will be apparent to those of skill in the art.

Genetically non-encoded amino acids such as β-amino acids, γ-amino acids, non-encoded a-amino acids and D-amino acids are also known to inhibit and/or prevent non-specific degradation. Thus, the masking moiety may also comprise one or more genetically non-encoded amino acid. For instance, the masking moiety can be a peptide consisting of one or more D-amino acids, one or more β-amino acids or mixtures of D- and β-amino acids. Preferred masking groups according to this class include N-methylalanine (“Me-Ala”), D-alanine and β-alanine.

In addition, masking moieties can comprise biologically inert molecules. Such molecules include small molecules such as dyes and polymers such as biological and non-biological polymers. For instance, polyalkylene glycols such as polyethylene glycol can prevent the degradation of a linking moiety and inhibit the activity of a biologically active entity. Preferred polyethylene glycol masking moieties have average molecular weights of about 1000 Da, about 4000 Da, about 5000 Da, about 8000 Da, about 10000 Da or about 12000 Da. Other suitable biological and non-biological polymers include, but are not limited to copolymers of divinyl ether and maleic anhydride (DIVEMA) or 2-hydroxypropyl methacrylate (HPMA), and other polymers such as DNA and carbohydrates. The masking moiety can also be a polypeptide that prevents the degradation of the prodrug and inhibits or prevents the activity of the biologically active entity. Preferred polypeptide masking moieties are non-immunogenic polypeptides. For instance, suitable polypeptide masking moieties include albumin, immunoglobulins, or an antibody.

In other embodiments of the invention, the masking moiety itself can have biological activity or other therapeutic activity. Prodrug compounds wherein the masking moiety has activity (“dual prodrugs”) are described in detail below. Suitable active masking moieties include cytostatic and cytotoxic small molecules, other therapeutically active small molecules, therapeutically active polypeptides and other therapeutically active molecules known to those of skill in the art.

Since the masking moiety is link d to the linking moiety, the masking moiety should either include a reactive group that is complementary to the terminus of the linking moiety to which it will be linked or be modified to include such a group. For instance, if the masking moiety is to be linked to the amino terminus of the linking moiety, the masking moiety should include a reactive group that is capable of forming a covalent linkage with the amino terminus, such as a carboxyl group. The linkage formed should be stable to the conditions of use of the prodrug, e.g., in the serum. Complementary reactive groups that are capable of reacting with the carboxy and amino termini of peptides to form stable linkages that may be included in the masking moiety are described in more detail below in connection with the biologically active entities. The masking moiety may inherently include such a complementary reactive group, or may be modified to include a suitable complementary reactive group. The methods of modification will depend upon the identity of the masking moiety and will be apparent to those of skill in the art.

5.6 Biologically Active Entities

The biologically active entity can be any entity that has biological activity against tumors or target cells, or any entity that would derive an advantage from being selectively administered to a tumor or target cell. Preferred biologically active entities include those entities that are cytotoxic and/or cytostatic to tumors and/or target cells such as, for example TNFα, IFN-α, IFN-γ, IL-1, IL-2, IL-6, an IGF-1 antagonist, a lytic peptide, an antiangiogenic peptide, a thrombospondin-derived peptide, a substance P antagonist, TRAIL (Apo-2 ligand) and Fas ligand, and also constructs comprising intracellularly active agents and a transport peptide allowing or facilitating their uptake in cells. As will be recognized by those having skill in the art, in order to be formulated as a prodrug of the invention, the biologically active entity must either inherently include, or be modified to include, a reactive group which is complementary to, Le., able to react to form a covalent linkage with, the reactive group on the linking moiety to which it will be covalently attached. Typically, pairs of such complementary reactive groups include nucleophile/electrophile pairs, as are well known in the art.

In many instances, the biologically active entity will be covalently attached to the N- or C-terminus of the linking moiety. Suitable reactive groups complementary to the linking moiety amino terminus include, for example, carboxy groups, esters (including activated esters such as NHS-esters), acyl azides, acyl halides, acyl nitrites, aldehydes, alkyl sulfonyl halides, halotriazines, imidoesters, isocyanates, isothiocyanates, sulfonate sters, etc. Suitable reactive groups complementary to the linking moiety carboxy terminus Include, for example, amines, alcohols, alkyl halides, thiols, hydrazines, diazoalkanes, sulfonate esters, etc. Conditions for forming such covalent linkages under suitably mild reaction conditions are well known. Preferably, the linkage between the linking moiety and the biologically active entity is an amide. Conditions for linking molecules together having complementary amino and carboxy groups to form amide linkages are well-known (see, e.g., Merrifield, 1997, Methods Enzymol. 289:3-13). Specific linking chemistries are provided in the Examples section.

If the biologically active entity is a polypeptide, then the preferred linkage is an amide bond between the carboxy terminal amino acid residue of the linking moiety and a free amino group of the polypeptide. The free amino group can be, for instance, at the amino terminus of the polypeptide or at the side chain of an amino acid, e.g. a lysine residue. In instances where the biologically active polypeptide comprises a plurality of amino-containing side chains (e.g., a plurality of lysine residues), a plurality of linking moieties may be linked to a single biologically active polypeptide molecule. The molar ratio of linked linking moieties can be conveniently controlled by adjusting the biologically active entity:linking moiety molar ratio of the conjugation reaction. Specific cleavage of the linking moiety in the vicinity of target cells such as tumor cells releases the biologically active polypeptide modified with a leucine residue or a phenylalanine residue at each amine group from which a linking moiety is cleaved.

Alternatively, the linking moiety can be linked to any other reactive group of the biologically active entity. For instance, the linking moiety can be linked to a free carboxyl group of the biologically active entity at, for example, the carboxy terminus or at the side chain of an acidic amino acid of a polypeptide or at a free carboxy group of other entities. The linking moiety can also be linked to other reactive moieties in the polypeptide including sulfhydryl moieties and other moieties known to those of skill in the art that are capable of forming a bond to the linking group. Appropriate adaptor moieties for each reactive moiety will be apparent to those of skill in the art.

In some instances, a biologically active entity may include a reactive group that does not yield a suitably stable linkage with the linking moiety, or it includes a reactive group that is not complementary to the desired reactive group on the linking moiety. For example, both the biologically active entity and the desired point of attachment to the linking moiety may include carboxyl groups. In these instances, the reactive group on either the biologically active entity or the linking moiety may first be converted to a complementary reactive group through the use of a bifunctional adaptor molecule. For example, a suitable bifunctional adaptor molecule for linking a biologically active entity including a carboxyl group to the carboxy terminus of a linking moiety could be a diamino alkyl, such as 1,3 diamino propane. Such adaptor molecules are somewhat analogous to the previously described spacer moieties used to construct dual polarity linking moieties of the invention (illustrated in FIG. 3E). Molecules suitable for use as adaptor molecules will depend upon the identities of the reactive groups, and will be apparent to those of skill in the art.

Preferably, the biologically active entity is linked to the linking moiety via those reactive groups of the biologically active entity on which the biologically active entity can tolerate derivatization without significant loss of biological activity. Because the prodrugs typically liberate a leucyl derivative of the biologically active entity upon specific cleavage of the linking moiety, the liberated leucyl derivative of the biologically active entity should possess optimal activity. To determine the proper site of linkage and/or ratio of linking moieties to biologically active entities, leucyl derivatives of the biologically active entity can be prepared and assayed for functional activity in, for instance, cell-based assays such as those described in the examples below and other assays suitable for measuring the activity of the biologically active entity known to those of skill in the art. In addition, if the biologically active entity is modified to generate a complementary reactive group for condensation with the linking moiety, then the leucyl derivative of the modified biologically active entity is assayed for optimal activity.

Preferably, the prodrug itself is also assayed for optimal activity. The intact prodrug should possess little or no activity in a normal physiological environment while the activated, released biologically active entity should possess optimal activity. Assays for functional activity include those assays known to those of skill in the art for measuring the stability, activation, toxicity and therapeutic activity of the prodrug. The assays can be conducted in vivo using, for instance test animals, or in vitro using cell based assays known to those of skill in the art. Preferred assays include cell-based assays using tumor cell models known to those of skill in the art. Exemplary assays for the stability, activation, toxicity and therapeutic activity are presented in the examples below. For a given combination of masking moiety, linking moiety and biologically active entity, a number of prodrugs can be prepared by varying the ratio of masking/linking moiety and biologically active entity. When the prodrug is prepared by solid phase or solution phase techniques the site of attachment of the masking/linking moiety to the biologically active entity can also be controlled. The results of activity assays for leucyl derivatives of the biologically active entity, described above, can be used to determine potentially useful stoichiometries and/or sites of attachment prior to screening. Ideal prodrugs are those that display the optimal combination of stability, toxicity, activation and therapeutic activity according to assays suitable for the biologically active entity known to those of skill in the art.

5.6.1 Extracellularly Active Biologically Active Entities

In one important aspect, the biologically active entity of the prodrug is an extracellularly active biologically active entity. An extracellularly active biologically active entity is an entity that can exert its biological activity without having to enter a cell. Preferred extracellularly active biologically active entities are those molecules that cannot traverse the cell membrane by themselves. Extracellularly active biologically active entities include small molecules such as small molecule agonists and antagonists of extracellular receptors and other small molecules that act extracellularly. Extracellularly active biologically active entities also include peptides and polypeptides such as cytokines, peptidic hormones, antibodies, and other extracellularly active molecules known to those of skill in the art. Specific examples of polypeptides that can be formulated as prodrugs according to the present invention include, but are not limited to, TNFα, IFN-α, IFN-γ, IL-1, IL-2, IL-6, an IGF-1 antagonist, thrombospondin-1 derived peptides, a substance P antagonist, TRAIL (Apo-2 ligand) and Fas ligand.

5.6.1.1 Tumor Selective Prodrug of TNFα

In one preferred embodiment of this aspect of the invention, a prodrug selectively delivers active TNFα to target cells. TNFα can be linked to a masking moiety via any linking moiety of the present invention.

TNFα is produced by many different cell types as a 26 kDa (233 amino acids) integral transmembrane protein from which a 17 kDa (157 amino acids) mature TNFα is released into the extracellular medium by proteolytic cleavage of an Ala-Val bond between residues 76-77. Its potential use as an antitumor entity led to its purification, cloning and expression as a recombinant protein. Biologically active TNFα exists as a trimer in solution and each subunit is a polypeptide of 157 amino acids. Each mature TNFα trimer can interact with three receptor molecules at the interface of the subunits. TNFα mediates its activities by binding to specific receptors on the surface of the majority of mammalian cells. Receptor aggregation upon TNFα binding might be the mechanism for receptor activation In the target cells (Banner et al., 1993, Cell 73:431-445). For an extensive review on TNFα, see Sidhu and Bollon, 1993, Pharmacol. Ther. 57:79-128.

TNFα can be cytotoxic, and cytostatic, on many cell types In vitro. However, this antiproliterative effect is not restricted to tumor cells and many normal cells, such as endothelial cells, smooth muscle cells, adipocytes, fibroblasts and keratinocytes are also inhibited by TNFα. Sensitivity of human endothelial cells to TNFα is correlated with their rate of proliferation.

Multiple clinical studies (phase I and II) have been carried out with recombinant TNFα as an anticancer entity without major therapeutic effect. TNFα resistance and mainly TNFα-induced systemic toxicity are two major limitations for the use of TNFα as an antineoplastic entity. TNFα can be used in clinical trials only if injected locally or by isolated limb perfusion owing to the severe toxicity after systemic injection. Data from phase I clinical studies, in which TNFα was administered in a variety of schedules have shown that the common toxic side effects include fever, chills, rigor, fatigue, diarrhea, nausea, headache, and hypotension. Severe hypotension is the dose-limiting toxicity. No significant clinical antitumor effects were observed in phase II trials when TNFα was given as a single entity.

Although TNFα showed promise in the regression of tumors in mice, the toxicity of TNFα prevents therapeutic effect in humans. Tumor regression in mice requires a dose of approximately 400 μg/kg while humans can tolerate only 8 to 10 μg/kg before life threatening toxicities set in (Kramer, et al., 1988, Cancer Research 48:920-925). In addition, TNFα has a very short half-life of 20 minutes after injection into humans. TNFα is rapidly cleared from blood and taken up by the kidney and the liver. It is moreover sensitive to N-terminal endopeptidases that inactivate it rapidly (Nakamura and Komiya, 1996, Biol. Pharm. Bull. 19:677-677). In vivo, animal studies have shown that like in vitro, other cytokines (IFN-α, IFN-γ, IL-1, IL-2 and IL-6) as well as cytotoxic drugs (cyclophosphamide, doxorubicin) enhance the antitumor action of TNFα. However, the toxic side effects increase to an unacceptable level and this does not solve the problem of inefficiency.

Conjugation of TNFα to biological and non-biological polymers has shown modest improvement in the stability of TNFα. For instance, TNFα can be conjugated to gelatin using carbodiimide. It retains 57% of its cytotoxic activity and is more active against tumors in vivo (Tabata et al., 1993, J. Pharm. Pharmacol. 45:303-308). TNFα was also coupled to polyethylene glycol via an amide bond between a lysine amino residue of TNFα and a terminal succinate group of PEG (N-succinimidyl succinate monomethoxy polyethylene glycol of a molecular weight of 5000). Although extensive PEG modification resulted in the complete loss of activity In vitro, conjugates partially modified retained a portion of the activity of TNFα. TNFα has also been conjugated to a divinyl ether and maleic anhydride copolymer (DIVEMA) of a molecular weight of 30,000.

A recent study has shown that chemical modification of TNFα with PEG increases the stability and antitumor potency of the cytokine (Tsutsumi et al., 1995, J. Pharmacol. and Exp. Therapeutics 278:1006-1011). TNFα was covalently coupled to PEG polymers and separated into fractions of various molecular sizes (Tsutsumi et al., 1995, supra). Increasing amounts of PEG relative to TNFα significantly reduced the specific activity of the TNFα (Tsutsumi et al., 1995, supra). However, the PEG conjugates displayed improved pharmacokinetic stability when compared to native PEG (Tsutsumi et al., 1995, supra). In addition, the PEG-TNFα conjugates showed greater tumor distribution in vivo than native TNFα (Tsutsumi et al., 1995, supra). Overall, the most effective PEG-TNFα conjugate, which had 56% of its lysine amino residues coupled to PEG, showed a 100-fold improved antitumor potency in vivo when compared to native PEG despite a 2-fold reduction in specific activity (Tsutsumi et al., 1995, supra). This derivative has also very striking activity against TNFα resistant tumors (Tsutsumi et al., 1995b, British J. of Cancer 71:963-968; Tsutsumi et al., 1996, J. Pharm. Exp. Therap. 278:1006-1011; Tsutsumi, 1996b, Jpn. J. Cancer Res. 87:1078-1085). The PEG-TNFα conjugate with 56% of its lysine residues modified showed the optimal balance of bioactivity, plasma stability and tissue distribution among the conjugates tested but was still too toxic to be used (Tsutsumi et al., 1995, supra). Although conjugates with more PEG modification showed improved stability, the overall potencies of the conjugates were reduced due to significant or total loss of specific activity. Later studies showed that with PEG of higher molecular weight (12,000) the TNFα-PEG conjugate lost most of its activity with a degree of lysine modification of only 36%. This greater loss is explained by a greater steric hindrance, preventing more easily TNFα from interacting with its receptor (Tsutsumi et al., 1996c, British J. Cancer 74:1090-1095).

To overcome the deficiencies of TNFα as an antineoplastic entity, TNFα can be formulated as a prodrug according to the present invention. A TNFα prodrug should display comparable plasma stability and tissue distribution to the PEG-TNFα conjugates described above. However, since selective activation of the prodrug at or near tumors or target cells liberates leucyl-TNFα, the prodrug conjugates should display little or no loss of TNFα specific activity in vivo. As a result, the prodrug can carry as much PEG as necessary to achieve complete inactivation, ensuring reduced toxicity and allowing the use of higher dose levels. In addition, selective activation of the prodrug at or near target cells should dramatically enhance the selectivity of the prodrug relative to the PEG-TNFα conjugates. The PEG-TNFα conjugates displayed only a mod St tissue distribution preference for tumor cells whereas the TNFα prodrug is selectively activated at or near tumor cells.

In one embodiment of this aspect of the invention, TNFα is formulated as a prodrug in which the masking moiety is a biocompatible polymer such as a polyalkylene glycol, preferably polyethylene glycol (PEG). PEG molecules of various average molecular weights can be used such as 1000 Da, 4000 Da, 5000 Da, 8000 Da, 10000 Da, 120000 Da or even higher. The linking moiety can be any linking moiety according to the present invention. Preferred linking moieties include Ala-Leu-Ala-Leu (SEQ ID NO:1), Leu-Ala-Leu-Ala-Leu (SEQ ID NO:2), Leu-Ala-Leu (SEQ ID NO:3), Leu-Ala (SEQ ID NO:4) or Leu-Ala-Phe (SEQ ID NO:5). Because TNFα includes several free amino groups, multiple different masking moieties and linking moieties can be used simultaneously.

The degree to which the free amino groups of the TNFα are saturated with PEG-masked linking moieties will depend upon, among other factors, the average molecular weight (MW) of the PEG (or other masking polymer). Generally, the higher the MW of the polymer or PEG, the lower the degree of saturation that is required to inactivate the activity of the TNFα in the prodrug formulations. Specific levels of saturation for a variety of PEGs of varying MWs are taught in Tsutsumi et al., 1995, supra.

Those of skill in the art will recognize that a particular advantage of the TNFα prodrugs of the invention is the ability to use a considerably higher level of saturation than that reported by Tsutsumi et al., 1995, supra. Because the PEG or polymer masking moieties are specifically and selectively cleaved at or near a tumor or target cell, when formulated as a prodrug according to the invention, the TNFα can be completely inactivated by the PEG or polymer masked linking moieties. Thus, unlike the TNFα-PEG complexes described by Tsutsumi et al., 1995, supra, regardless of the MW of the PEG used, the TNFα molecule may be saturated with PEG-masked linking moieties. Thus, the PEG-TNFα prodrugs of the invention, in addition to being selective, may exhibit even lower toxicities than the PEG-TNFα complexes described in the art.

Specific PEG-TNFα prodrugs of the invention, as well as methods for their synthesis, are provided in the Examples.

5.6.1.2 Tumor Activated IGF-1 Antagonist Prodrug

In another embodiment, a prodrug selectively delivers an oligopeptide antagonist of insulin-like growth factor (IGF-1) to tumors or target cells in vivo. The masking moiety can be selected from any of the masking moieties discussed above, and the linking moiety can also be selected from any of the linking moieties discussed above. In a preferred embodiment the masking moiety is a polymer such as PEG, more preferably a succinylated derivative of PEG, and the linking moiety is the tetrapeptide Ala-Leu-Ala-Leu. The linking moiety is linked to free amino groups of the antagonist, either at one or more lysine side chains and/or at the amino terminus.

The expression of insulin-like growth factors and their type I receptor is very often up-regulated during the development of many cancer types. Recent data show that, besides stimulating cell transformation and tumor cell cycle progression, signaling through the IGF-1 receptor exerts an important tumor promotion effect by inhibiting tumor cell apoptosis (including drug-induced apoptosis). Consistently, inhibition of the IGF-mediated survival function is likely to increase the antitumor effects of conventional chemotherapy (Gooch J. L. et al., 1999, Breast Cancer Res. Treat. 56:1-10).

Antagonists of IGF-1 composed wholly of D-amino acids have been designed which resemble IGF-1 receptor ligands and efficiently inhibit IGF-1 receptor function in vitro and in vivo. One such peptide is D-Cys-D-Ser-D-Lys-D-Ala-D-Pro-D-Lys-D-Leu-D-Pro-D-Ala-D-Ala-D-Tyr-D-Cys which is a retro-enantio sequence derived from the D domain of IGF-1. In order to further increase the overlap between the conformation of the native protein with that of the retro-enantio peptide, it has been cyclized via an artificially introduced disulfide bridge (Hayry et al., 1995, Faseb J. 9:1336-1344; Pietrzkowski et al., 1992, Cancer Res. 52:6447-6451).

Such antagonists could become very useful anticancer entities, but because of the crucial importance of IGF-1 receptor-mediated signaling in many normal cells and tissues, as well as the high similarity between the IGF-1 receptor and the insulin receptor, such antagonists must be administered selectively to cancer cells to avoid toxic side effects.

In one embodiment of the invention PEG is linked to the IGF-1 antagonist via the tetrapeptide linker Ala-Leu-Ala-Leu. The antagonist is derivatized only on those reactive groups that can be derivatized with leucine without significant loss of antagonist activity.

5.6.1.3 Tumor Activated Prodrug of a Thrombospondin-1 Derived Peptide

In another preferred embodiment, a prodrug compound is capable of selectively delivering to target cells an antiangiogenic peptide derived from the structure of the angiogenesis inhibitor thrombospondin-1 (TSP-1).

The continuous growth and metastasis of all tumors is strongly dependent on neoangiogenesis. In experimental systems, inhibitors of angiogenesis have been shown to be remarkably active anticancer entitles towards which tumors do not seem to develop resistance (Harris, 1997, The Lancet 349:13-15; Dawson et al., 1999, Mol. Pharmacol. 55:332-338; Bcehm et al., 1997, Nature 390:404-407). There remains, however, a need to develop new antiangiogenic drugs with improved potency, stability, and selectivity (Molema et al., 1998, Biochem. Pharmacol. 55:1939-1945). Antiangiogenic peptides have been developed consisting partly or entirely of D-amino acids (Dawson et al., 1999, supra).

Thrombospondin-1 (TSP-1) is a naturally occurring inhibitor of angiogenesis and provides one source for the development of new anti-angiogenic molecules. TSP-1 is a 450-kDa homotrimeric protein with multiple structural domains that contribute to its involvement in diverse biological activities including angiogenesis. The therapeutic potential of TSP-1 has been demonstrated in animal models where it has been shown to block the growth and progression of malignant tumors by hindering neovascularization.

In this embodiment of the invention, peptides derived from the primary sequence of TSP-1 are formulated as prodrug compounds for selective delivery to target cells. In particular, peptide sequences that comprise reverse sequences of D-amino acids derived from a type-I repeat of amino acids from the primary structure of TSP-1 have antiproliferative and antiangiogenic properties (Dawson et al., 1999, supra). Such peptides are preferred for formulation as prodrug compounds in this embodiment of the invention. In this embodiment of the invention, preferred masking moieties include biocompatible polymers such as PEG, and preferred linking moieties include Ala-Leu-Ala-Leu (SEQ ID NO:1), Leu-Ala-Leu-Ala-Leu (SEQ ID NO:2), Leu-Ala-Leu (SEQ ID NO:3), Leu-Ala (SEQ ID NO:4) or Leu-Ala-Phe (SEQ ID NO:5).

5.6.1.4 Tumor Activated Prodrug of a Substance P Antagonist

In another preferred embodiment of the invention, substance P antagonists are formulated as prodrug compounds for selective delivery to target cells.

Growth factors play an important role in the pathogenesis and evolution of cancers. New targets for therapy have been identified from the knowledge of the role such growth factors play in the progression of lung cancer. Peptide antagonists of bombesin, bradykinin and substance P have been developed by substituting D-amino acids in fragments of the corresponding native growth factor. The antagonists block the biological effects of a broad range of neuropeptides and inhibit small-cell lung (SCLC) and non-small-cell lung cancer (NSCLC) cell proliferation in vitro, as well as in vivo (Bunn et al., 1994, Cancer Res. 54:3602-3610; Secki et al., 1997, Cancer Res. 57:51-54; Chan and Geraci, 1998, Drug Resistance Updates 1:377-388). In particular, substance P antagonists have been shown to induce apoptosis in SCLC cells through a currently unknown mechanism (Chan and Geraci, 1998, supra).

Accordingly, in this embodiment, substance P antagonists are formulated as prodrugs for selective delivery to target cells. A preferred substance P antagonist is a peptide with the amino acid sequence D-Arg-Pro-Lys-Pro-D-Trp-Gln-D-Trp-Phe-D-Trp-Leu-Leu-NH₂ (“SPD”). Preferred masking moieties include biocompatible polymers such as PEG, and preferred linking moieties include Ala-Leu-Ala-Leu (SEQ ID NO:1), Leu-Ala-Leu-Ala-Leu (SEQ ID NO:2), Leu-Ala-Leu (SEQ ID NO:3), Leu-Ala (SEQ ID NO:4) or Leu-Ala-Phe (SEQ ID NO:5).

5.6.2 Polypeptide

In other embodiments of the invention, the biologically active entity can be a polypeptide. Any cytotoxic and/or cytostatic polypeptide can be formulated as a prodrug according to the present invention. When the biologically active entity is a polypeptide, preferred linking moieties include peptides and the preferred linkage between the polypeptide biologically active entity and the linking moiety is an amide bond. Suitable polypeptides include the extracellularly active polypeptides described above including TNFα, IFN-α, IFN-γ, IL-1, IL-2, IL-6, an IGF-1 antagonist, thrombospondin-1 derived peptides, a substance P antagonist, TRAIL (Apo-2 ligand) and Fas ligand. Polypeptides suitable for these embodiments of the invention also include polypeptides that are not necessarily extracellularly active. Polypeptide biologically active entities include intracellularly active polypeptides such as, for example, granzyme B and other polypeptides with diverse functions such as, for example, lytic peptides.

5.6.2.1 Tumor Activated Prodrug of a Lytic Peptide

In another embodiment of the invention, a prodrug selectively delivers to target cells a peptide that is capable of lysing those cells in vivo. In this embodiment, a lytic peptide is linked to any of the masking moieties discussed supra via any of the linking moieties discussed supra. The linking moieties can be linked to any reactive group of the lytic peptide such as free amino groups. Preferred masking moieties include PEG and preferred linking moieties include the tetrapeptide Ala-Leu-Ala-Leu. A succinylated PEG derivative can conveniently be linked to the linking moiety.

Melittin, a 26-residue peptide found in the venom of the European honey bee is cytolytic. Its activity is dependent on the amphipathicity of the alpha-helix formed by its first 20 amino acids (D mpsey, 1990, Biochim. Biophys. Acta 1031:143-161; Werkmeister et al., 1993, Biochim. Biophys. Acta 1157:50-54, 1993). On the basis of the amphipathic alpha-helical structure of melittin, simpler sequences have been designed that have similar or greater cytolytic activity (Cornut et al., 1994, FEBS Lett. 349:29-33; Castano et al., 1999, Biochim. Biophys. Acta 1416:161-175). This is the case of alpha-helices made only of alternating lysine and leucine residues (5 to 22-mers; KᵢLⱼ, j=2i). A length of 15 residues seems to be optimal in the experimental conditions used in vitro in the absence of serum (Lys-Leu-Leu-Lys-Leu-Leu-Leu-Lys-Leu-Leu-Leu-Lys-Leu-Leu-Lys). Furthermore, blocking the N-terminal alpha-amino group has been shown to improve efficacy of the peptide.

A similar sequence made of D-lysine and D-leucine residues (LK15) forms an amphipathic alpha-helix with similar cytolytic properties. It has the advantage of resistance to the action of most mammalian peptidases found in body fluids, and is therefore much more stable in vivo.

The action of the lytic peptide on tumor cell membranes could overcome drug resistance directly (lysis), by enabling a cytotoxic drug to enter cells to a greater extent, or by disrupting metabolic processes involved in the resistance mechanism.

Due to their intrinsic cytolytic activity, naked lytic peptides active on mammalian cells are inherently toxic. They must be targeted specifically to tumor cells in the form of prodrugs.

In a preferred embodiment, the prodrug comprises the lytic peptide LK15 linked to the masking moiety PEG via the tetrapeptide linking moiety Ala-Leu-Ala-Leu. The lytic peptide is derivatized only on those reactive groups that can tolerate leucyl-derivatization without significant loss of lytic activity.

5.6.3 Intracellularly Active Prodrugs

In another preferred embodiment, the biologically active entity is a construct comprising an intracellularly active entity linked to a transport peptide. The intracellularly active entity is cytotoxic or cytostatic toward cells and exerts its cytotoxic or cytostatic activity within the cell. Prodrug compounds comprising intracellularly active entities are illustrated by Formula (II):

(M−L¹)ₚ−TP−(L²−A)ₘ (II)

wherein M and L¹ are as defined In Formula (I) above, TP Is a transport peptide, L² is an optional intracellularly labile cleavage site, A is an Intracellularly active biologically active agent, m is an integer from 1 up to (k−1) and p is an integer from 1 up to (k−m), where k is an integer equal to the total number of reactive groups of TP.

Suitable intracellularly active entities include intracellularly active small molecules, peptides, proteins, nucleic acids or analogs thereof. These entities may or may not be able to penetrate the cells by themselves. For example, suitable intracellularly active entities include small molecules capable of penetrating cells such as anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecins, proteasome inhibitors, farnesyl-protein transferase inhibitors, epothilones, maytansinoids, discodermolide, platinum derivatives, duocarmycins, combretastatin and epipodophyllotoxins. Intracellularly active entities also include larger molecules that are unable to penetrate cells or inefficiently penetrate cells such as antisense nucleic acids including antisense RNA and DNA, ribozymes, DNA, cDNA, genes, proteins and polypeptides.

The transport peptide portion of the construct enables, facilitates or enhances transport of the intracellularly active entity into the target cell and/or nuclear translocation of the entity. The action of any biologically active entity, including intracellularly active biologically active entities can potentially be improved by formulation as a prodrug with a transport peptide construct. For instance, formulation of an intracellularly active biologically active entity, such as doxorubicin, as a prodrug with a transport peptide construct may not only improve the uptake of the entity, it may also allow delivery of the entity to the nucleus of the cell. Improved nuclear delivery of the entity that is active in the nucleus would increase its efficiency of action and would allow reduced resistance to the entity.

Four distinct types of peptides have been recently characterized that have the unusual property of being transported into the cell and/or into the nucleus, and to carry with them, complexed or conjugated peptides, proteins, nucleic acids, as well as small molecules. Three of these types of peptides, the Antennapedia homeodomain-derived peptide, the HIV Tat transactivation protein-derived peptide, and 9-mers of arginine enter cells by translocating through the plasma membrane without disrupting it. They enter cells at 4° C. as well as at physiological temperature, and their uptake is not receptor-dependent. After crossing the cell membrane they reach the cytoplasm and can be conveyed to the nucleus (PCT Application No. WO98/52614; Derossi et al., 1996, J. Biol. Chem. 271:18188-18193; Vives et al., 1997, J. Biol. Chem. 272:16010-16017).

The Antennapedia peptide is a 16-amino acid polypeptide (Lys-Lys-Trp-Lys-Met-Arg-Arg-Asn-Gln-Phe-Trp-Val-Lys-Val-Gln-Arg-Gly) corresponding to the third helix of the DNA-binding homeodomain of Antennapedia, a Drosophila transcription factor (Derossi et al., 1994, J. Biol. Chem. 269:10444-10450). The translocation process seems to be based on the establishment of direct interactions between the positively charged peptide and the negatively charged membrane phospholipids followed by the induction of inverted micelles. The hydrophilic cavity of the micelles accommodates the peptide that can subsequently be released in the cytoplasmic compartment (Derossi et al., 1996, supra). This peptide has been used in vitro to carry intracellularly into cancer cells a CDK inhibitor and a p53-derived peptide (Bonfanti et al., 1997, Cancer Res. 57:1442-1446; Kim et al., 1999, J. Biol. Chem. 274:34924-34931).

Tat is a 86-amino acid protein involved in the replication of HIV-1. Exogenous Tat protein translocates through the plasma membrane and reaches the cell nucleus. This translocation activity has been assigned to a cluster of basic amino acids, and short peptides including this cluster can translocate through the plasma membrane as well, and convey proteins to the nucleus. One example of such a short and very effective peptide is Gly-Arg-Lys-Lys-Arg-Arg-Gln-Arg-Arg-Arg-Pro-Pro-Gln-Cys. Translocation also occurs at 4° C. and does not involve endocytosis or recognition by a membrane receptor. The mode of translocation seems to be similar to that of the Antennapedia peptide and involves the interaction of the basic amino acids with the negatively charged phospholipids of the cell membrane (Vives et al., 1997, supra).

Polypeptides consisting of 6 to 25 residues, at least 50% of which contain a guanidino or an amino moiety, are also able to translocate across cell membranes compounds such as small organic molecules, peptides, proteins and nucleic acids at 4° C. as well as at physiological temperature. Particularly, peptides consisting of 9 contiguous arginine residues were shown to very efficiently translocate into every cell type tested antisense peptide nucleic acids, ovalbumin, antibodies, diverse peptides, cyclosporin and taxol. The precise mechanism of translocation is still unknown (PCT Application No. WO98/52614).

The fourth class of peptides (from 3 to 30 amino acid residues in length) are derived from the CDR regions of polyreactive anti-DNA antibodies occurring naturally or pathologically in mice and humans. An example of a 30 amino acid transport peptide derived from an anti-DNA antibody is Val-Ala-Tyr-Ile-Ser-Arg-Gly-Gly-Val-Ser-Thr-Tyr-Tyr-Ser-Asp-Thr-Val-Lys-Gly-Arg-Phe-Thr-Arg-Gln-Lys-Tyr-Asn-Lys-Arg-Ala. These peptides do not penetrate cells at 4° C. and are likely taken up by endocytosis (Avraméas et al., 1998, Proc. Natl. Acad. Sci. USA 95:5601-5606; PCT Application No. WO99/07414). By a still unidentified mechanism, the peptides are translocated to the cytoplasm and nucleus. They can also convey proteins, enzymes and nucleic acids to the nucleus.

Versions of these transport peptides made partly or entirely of D-amino acids can also be used. In vivo, they will have the advantage of a much improved stability.

In this embodiment a masking moiety can be any moiety that inhibits the biological activity of the intracellularly active entity by preventing the translocation of the transport peptide-intracellularly active entity construct across cell membranes, and that prevents the non-specific in vivo degradation of the optional linking moiety. When the prodrug is in the environment of a healthy cell that displays little or none of the conditions that are capable of cleaving the masking moiety and/or the linking moiety, the masking moiety prevents non-selective entry of the intracellularly active entity into the healthy cell. The toxicity of the prodrug is thereby reduced. In the environment of a tumor or target cell, the masking and/or the linking moiety is cleaved liberating the construct. The transport peptide portion of the construct carries the intracellularly active entity into the cell where the entity can exert its activity on the tumor or target cell.

The construct can optionally comprise a cleavage site between the transport peptide and the intracellularly active entity. The cleavage site must be susceptible to cleavage within a cell and resistant to cleavage in the extracellular milieu or in the serum. The cleavage site can itself be resistant to extracellular cleavage, or the cleavage site can be resistant to extracellular cleavage when the construct is formulated as a prodrug. Because selective cleavage of the linking moiety of the prodrug provides selective activation of the construct at or near target cells, the cleavage site need not be selectively cleavable at or near target cells. However, the cleavage site can be optionally susceptible to selective cleavage within a target cell to impart even better activity and even greater target cell selectivity to the prodrugs of this embodiment. Suitable cleavage sites include peptide sequences that are stable in the extracellular environment and susceptible to protease cleavage within a cell. For instance, suitable protease-sensitive peptide sequences include but are not restricted to Arg-Xaa-(Lys/Arg)-Arg or other furin substrates (Nakayama, 1997, Biochem. J. 327:625-635), Asp-Glu-Val-Asp-Ala-Pro-Lys or other caspase substrates (Enari H. et al., 1996, Nature 380:723-726) and Leu-Leu-Val-Tyr or other proteasome substrates (Rock et al., 1994, Cell 78:761-771).

5.6.3.1 Masked Derivatives f a Transport Peptide

In an embodiment of the invention, a transport peptide is selectively masked with a masking moiety via a linking moiety. The masking moiety, alone or together with the linking moiety, prevents intracellular transport of the transport peptide. When the linking moiety is selectively cleaved, the active transport peptide is liberated. The transport peptide can then carry a biologically active entity with it into a cell and/or into the nucleus of the cell. The linking moiety can be any of the linking moieties discussed supra, and the masking moiety can be any of the masking moieties discussed supra. The linking moiety can be linked to any reactive group of the transport peptide that can tolerate leucyl derivatization without significant or complete loss of transport activity. Preferred masking moieties include succinylated PEG and preferred linking moieties include the tetrapeptide Ala-Leu-Ala-Leu.

These transport peptides have the unusual property of being transported intracellularly and into the nucleus. They are able to carry with them complexed or conjugated peptides, proteins, nucleic acids, and small molecules. However, the transport peptides are transported into a broad range of cell types, including most if not all normal cells. A transport peptide construct comprising a cytotoxic or cytostatic entities therefore suffers severe toxicity problems.

Selective delivery of a transport peptide construct to target cells that would spare normal cells, thereby minimizing toxicity, is particularly desirable. In order to develop cancer cell-selective vectors, the transport peptide construct is formulated as a prodrug according to the present invention. The transport activity of the peptide is reversibly blocked, for instance, by conjugation to a masking moiety via the linking tetrapeptide Ala-Leu-Ala-Leu. The activity is blocked due to conjugation on or close to specific, essential positively charged side chains of the transport peptides. The linking tetrapeptide is cleavable by peptidases released selectively in the extracellular environment of target cells. For example, modification of the transport peptide with PEG masks the transport activity of the peptide and also increases the solubility and half-life of the prodrug.

Since selective cleavage of the prodrug liberates a leucine derivative of the transport peptide, residues of the transport peptide that can be derivatized with minimal loss of transport activity are identified. Reactive groups of the transport peptide are first derivatized with leucine residues to determine appropriate sites for derivatization with a linking moiety and a masking moiety. Appropriate reactive groups of the transport peptide include any reactive group that can tolerate derivatization with a leucine residue without significant loss of transport activity.

An exemplary prodrug according to this embodiment of the present invention comprises doxorubicin linked to a derivatized transport peptide. The transport peptide can be derivatized with the masking moiety PEG via the linking tetrapeptide Ala-Leu-Ala-Leu at the appropriate reactive groups of the transport peptide determined as described above.

5.6.3.2 Pro-Apoptotic Protein—Carrier Peptide Prodrug

In this embodiment of the present invention, a prodrug selectively transports an intracellularly active, pro-apoptotic protein into target cells in vivo. A pro-apoptotic protein is formulated in a prodrug as a construct comprising the pro-apoptotic protein and a transport peptide. The transport peptide facilitates entry of the pro-apoptotic protein into the cell. The transport peptide is chosen from those described above. The transport peptide can be linked to any reactive group of the pro-apoptotic protein, and preferred reactive groups include the free amino group at the amino terminus of the protein and free ε amino groups of lysine side chains of the protein. A preferred apoptotic protein in this embodiment of the invention is Granzyme B.

The masking moiety can be any masking moiety described above that, alone or together with the linking moiety, prevents the transport activity of the peptide and that prevents the non-specific cleavage of the linking moiety. The linking moiety can be any linking moiety described supra. Preferred masking moieties include PEG, preferred linking moieties include the tetrapeptide Ala-Leu-Ala-Leu. When the prodrug is in the environment of a target cell, the linking moiety is cleaved liberating an active leucyl derivative of the active transport peptide. The transport peptide carries with it the pro-apoptotic protein into the cell. The transport peptide can be linked to the protein via an optional cleavage site susceptible to cleavage within the cell. If so, the cleavage site can be cleaved within the cell liberating the intact, active pro-apoptotic protein within the target cell.

Granzyme B, a single-chain serine protease of about 28.5 kDa, was first demonstrated to play a crucial role in the initiation of apoptosis induced by killer lymphocytes. This killing effect results from the synergistic effect of perforin, a membranolytic protein and the serine protease granzyme B (Blink et al., 1999, Immunol. Cell Biol. 77:206-215; Trapani et al., 1998, J. Biol. Chem. 273:27934-27938). Perforin allows granzyme B to reach the cytoplasm and the nucleus of cells by inducing the formation of transmembrane pores that constitute a passage for the enzyme. Granzyme B then induces apoptosis by starting pre-existing death pathways through the enzymatic cleavage and activation of pro-caspases, and also by directly cleaving nuclear substrates such as DNA-PK and poly-ADP ribose polymerase (Froelich et al., 1996, Biochem. Biophys. Res. Commun. 227:658-665; Yang et al., 1998, J. Biol. Chem. 273:34278-34283). In the prodrug, the transport peptide potentially plays the role of perforin by allowing granzyme B to enter the cell and to induce apoptosis.

Preferred prodrugs of this embodiment of the present invention comprise granzyme B linked to the PEG-Ala-Leu-Ala-Leu derivative of the transport peptide, described above, via an amide bond from the carboxy terminus of granzyme B to the amino terminus of the derivatized transport peptide. The granzyme B prodrug can be administered alone or in combination with doxorubicin or any doxorubicin prodrug described above.

5.7 Dual Prodrug Compounds

In important embodiments of the invention, the prodrug compounds are dual prodrugs. A dual prodrug compound can deliver two or more entities to target cells. In dual prodrugs, the biologically active entity is a cytostatic or cytotoxic entity as described in detail above. In addition, the masking moiety of the dual prodrug also has biological activity.

The masking moiety can be biologically active intracellularly or extracellularly so long as the masking moiety, alone or together with the linking moiety, prevents the activity of the biologically active entity, is inactive prior to its release from the prodrug, and prevents the in vivo degradation of the prodrug. Suitable masking moieties can be selected from the extracellular and intracellular biologically active entities discussed in detail herein. Suitable masking moieties also include small molecule therapeutic entities. For instance, suitable masking moieties can be selected from anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecin, proteasome inhibitor, farnesyl transferase inhibitor, epothilones, maytansinoids, discodermolide, platinum derivatives, duocarmycins, combrestastatin and epipodophyllotoxins.

Dual prodrugs of the present invention include any pair of entities that have a biological activity and a therapeutic effect on a tumor or target cell. For instance, a dual prodrug can comprise two biologically active polypeptides, two biologically active small molecules, two extracellularly active biologically active entities, a biologically active polypeptide and a biologically active small molecule, and other pairs of biologically active entities apparent to one of skill In the art. Particularly useful prodrugs of the present invention are those that comprise a pair of entities that act in concert at a target cell. For instance, one of the entities can be a ligand for a cell surface receptor that facilitates transport of the other entity when bound to the receptor. In another useful pair, one entity can alter the permeability of the cell membrane to facilitate transport of the other entity. In a third pair, a small molecule and a polypeptide can have synergistic effects on the same target cell.

The masking moiety and biologically active entities may be linked to the linking moiety as previously described (see, e.g., FIG. 3B, FIG. 3C, and FIG. 3D). If neither the biologically active entity nor the masking moiety retains sufficient activity following cleavage when linked to the amino terminus of the linking moiety, they must both be linked to the linking moiety via carboxyl groups. This can be conveniently achieved using a “dual polarity” linking moiety as illustrated in FIG. 3E.

Referring to FIG. 3E, dual polarity linking moiety 58 is linked to two biologically active entities 38 to yield dual prodrug 52. Dual polarity linking moiety 58 comprises three segments: a first segment 53, a second segment 55 and a third segment 57. Segments 53 and 57 are each linking moieties as described herein, and may be the same or different. Linking moiety segments 53 and 57 are linked together via second segment 55 at their amino termini. Thus, second segment 53 is typically a spacer or an adaptor moiety having two reactive groups capable of forming a covalent linkage with an amino group, such as a dicarboxylic acid (e.g., citraconyl, dimethylmaleyl, glutaryl, succinyl and diglycolyl). When placed in the vicinity of a target cell, dual prodrug 52 is cleaved to release 2 moles of released biologically active entity 39 for every mole of dual prodrug 52. Of course, dual prodrug 52 may optionally include spacing moieties intervening one or both biologically active moieties, as previously described.

In the dual prodrug 52 illustrated in FIG. 3E, linking segments 53 and 55 are identical, as are biologically active entities 38. However, those of skill in the art will recognize that they need not be. Linking segments 53 and 55 and biologically active entities 38 may each be, independently of one another, the same or different.

In certain dual prodrug embodiments of the invention, one of the biologically active entities is the small molecule active entity doxorubicin. Doxorubicin can be linked to any of the biologically active agents discussed supra. For instance, in one embodiment a dual prodrug delivers the two antineoplastic entities, TNFα and doxorubicin. In two other dual prodrug embodiments, doxorubicin can be linked to the IGF-1 antagonist described supra or to the lytic peptide LK15 described supra. One doxorubicin molecule can even be linked to another doxorubicin molecule via on of the linking moieties of the present invention to form a selective prodrug.

5.7.1 Dual Prodrugs Comprising TNFα

Preferred dual prodrugs comprise pairs of biologically active agents that act in concert with each other. For instance, the cytotoxic effect of TNFα on many tumor cell lines is enhanced by other cytokines and antitumor drugs. For instance, IFN-γ, IFN-α and IL-1 have been shown to enhance the cytotoxic effects of TNFα or to abrogate the cellular resistance to TNFα. There is convincing evidence that there is a synergy between TNFα and topoisomerase-targeted drugs such as doxorubicin, VM-26, etoposide, teniposide and daunorubicin. This synergy is related to a rapid increase in specific activity of topoisomerase I and II resulting in enhanced DNA strand breaks and cleavage complexes (Kreuser et al., 1995, Recent Results Cancer Res. 139:371-382). Doxorubicin can also suppress the resistance of tumor cells to TNFα, due to endogenous TNFα, provided it is administered before or during the treatment with TNFα (Borsellino et al., 1995, Anticancer Res. 14:2643-2648; Watanabe et al., 1995, Jpn. J. Cancer Res. 86:395-399).

TNFα has been shown to act synergistically with classical anticancer entities, and particularly anthracyclines, a dual prodrug comprising TNFα and doxorubicin is prepared. Doxorubicin can be linked to the carboxy terminal end of a peptide linking moiety, and TNFα can be linked to the amino terminal end of the linking moiety via a dicarboxylic acid spacer. Activation of the prodrug in the vicinity of a target cell liberates leucyl-doxorubicin and TNFα modified with a portion of the linking moiety.

In one embodiment of the invention, the biologically active entity doxorubicin is linked to the tetrapeptide linking moiety Ala-Leu-Ala-Leu as described (U.S. Pat. No. 5,962,216) to yield Ala-Leu-Ala-Leu-Dox. A TNFα dual prodrug is prepared by linking Ala-Leu-Ala-Leu-Dox to a leucyl-derivative of TNFα via a methylmaleyl adaptor moiety.

5.7.2 Dual Thrombospondin-1 Derivative Prodrug

In other embodiments, doxorubicin can be linked to the thrombospondin-1-derived peptides, discussed supra, to generate potent dual prodrugs.

Since synergies have frequently been observed between antiangiogenic compounds and cytotoxic anticancer entities (Teicher et al., 1992, Cancer Res. 52:6702-6704), a combination of such a peptide and an anthracycline in the form of a dual prodrug could enhance the anti-tumor effect of each one of the two entities. It could also obviously help to overcome drug resistance problems.

In one embodiment of the Invention, dual prodrugs are prepared by linking Ala-Leu-Ala-Leu-Dox to the carboxy terminus or to amino acid side chains of peptide derivatives of TSP-1. The biologically active entities of this embodiment of the present invention comprise peptides derived from consensus sequences from the primary structure of TSP-1. In particular, the reverse sequences of D-amino acids derived from a type-I repeat of amino acids of TSP-1, which have antiproliferative and antiangiogenic properties (Dawson, et al., 1999, supra), are particularly useful biologically active entities in this embodiment of the present invention.

5.7.3 Dual Substance P Antagonist Prodrug

In further embodiments of the invention, dual prodrugs can be prepared by linking doxorubicin to a substance P antagonist.

Recent results have indicated that the use of substance P antagonists in combination with chemotherapeutic entities may provide a way to overcome drug resistance in lung cancer (Chan and Geraci, 1998, supra). Conjugating anticancer entities to antagonists of substance P growth factors provides an efficient approach for the development of potent drugs against small cell lung carcinoma and non-small cell lung carcinoma.

A dual prodrug according to this embodiment of the present invention comprises the potent substance P antagonist with the sequence D-Arg-Pro-Lys-Pro-D-Trp-Gln-D-Trp-Phe-D-Trp-Leu-Leu-NH₂ (“SPD”). Ala-Leu-Ala-Leu-Dox can be linked to the amino terminus of SPD via a dicarboxylic methylmaleyl moiety.

5.8 Methods of Making Prodrug Compounds

The prodrug compounds can be prepared according to standard synthetic or recombinant techniques known to those of skill in the art. For instance, peptide linking moieties can be synthesized by conventional solid phase or solution phase peptide chemistry. Biologically active entities and masking moieties can be obtained from commercial sources or from other well-known methods such as purification from natural sources, recombinant expression and other techniques. Dual polarity linkers and spacer moieties can be synthesized or obtained from commercial sources or from other well-known methods.

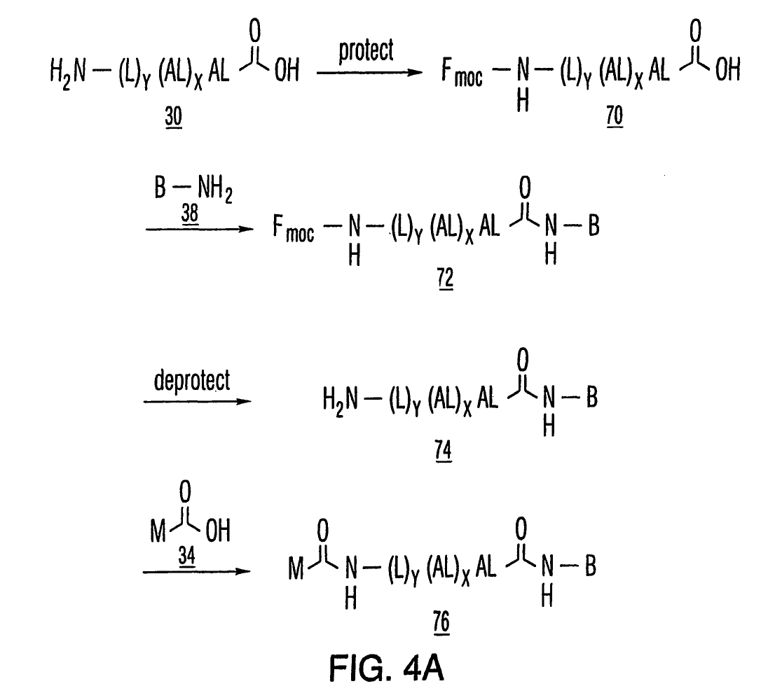

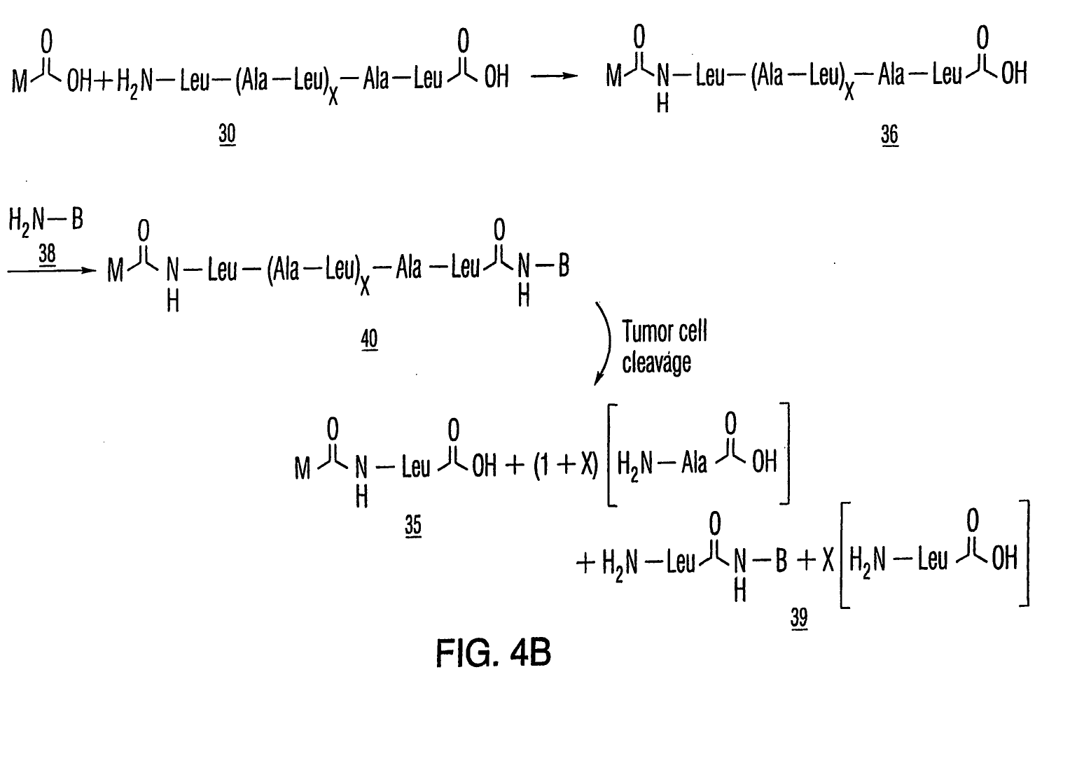

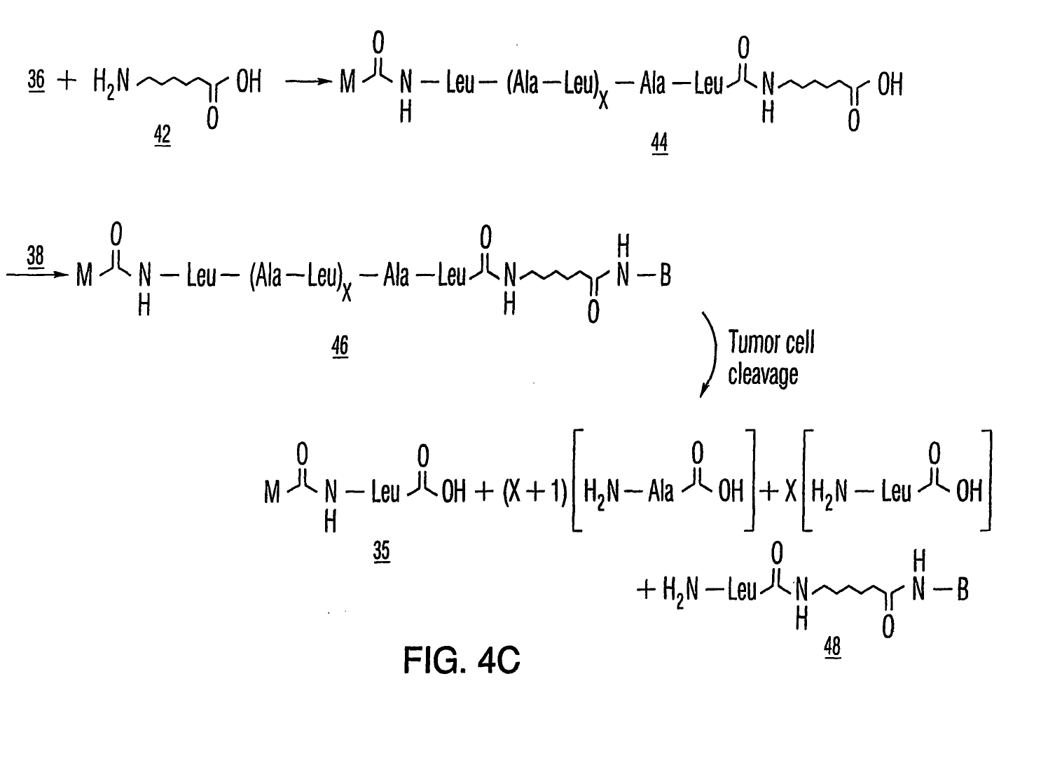

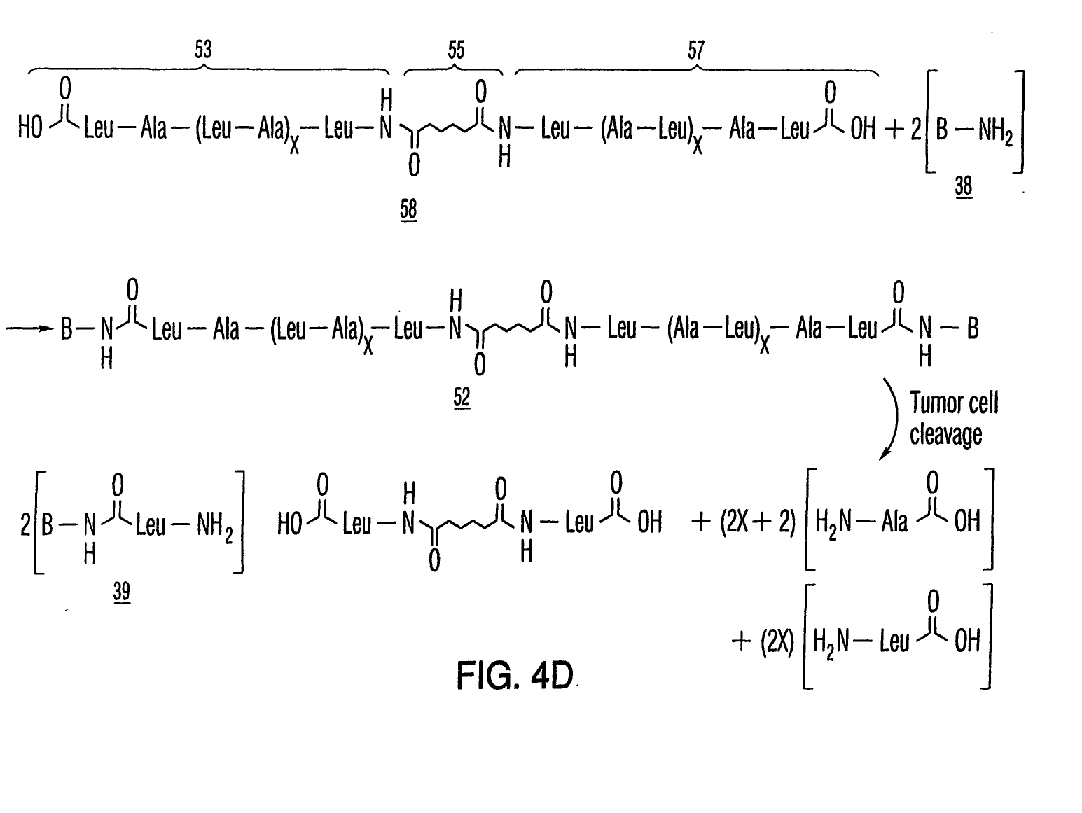

Typically, the prodrugs are prepared synthetically by condensing the masking moiety and biologically active entity with the linking moiety. Well known protecting groups can be used advantageously In the preparation of prodrug compounds. For example, a prodrug compound of the present invention can be prepared according to the scheme presented in FIG. 4A. The free amino group of linking moiety 30 is first protected with a standard protecting group such as Fmoc to yield Fmoc-protected compound 70. A covalent bond is then formed by condensing a reactive group of Fmoc-protected 70 with a complementary reactive group of biologically active entity 38 to form the Fmoc-protected complex 72. Deprotection of the complex 72 yields compound 74. Subsequent condensation with a complementary reactive group of masking moiety 34 forms the prodrug compound 76. One of skill in the art will recognize that this general scheme can be adapted for virtually any prodrug. For instance, the masking moiety can be linked to the linking moiety prior to reaction with the biologically active agent. Moreover, a plurality of Fmoc-protected complexes 72 may be condensed with a single biologically active entity 38 by routine adjustment of their molar ratios.

In FIG. 4B, an exemplary prodrug composition of the invention 40 is constructed by coupling an activated form of masking moiety 34 including a reactive carboxy group to the amino terminus of linking moiety 30 to yield compound 36. Compound 36 is linked to a biologically active entity 38 which includes a reactive amine group to yield prodrug 40. In the vicinity of target cells, prodrug 40 is specifically cleaved to yield Compound 35 and released biologically active entity 39. Released biologically active entity 39 includes the C-terminal leucine residue from linking moiety 30. To control the reaction, the protecting strategy of FIG. 4A may be used.

FIG. 4C illustrates the preparation of a prodrug of the invention which comprises a spacing moiety. Compound 36, which comprises masking moiety 34 linked to linking moiety 30, is coupled with spacing moiety 42 to yield compound 44. Compound 44 is then coupled with biologically active entity 38 to yield a prodrug compound wherein the biologically active entity 38 is separated from linking moiety 30 by spacing moiety 42. Activation of the prodrug in the vicinity of a target cell liberates compound 48 comprising biologically active entity 38, spacing moiety 42 and a leucine residue. Again, the synthesis may be controlled by employing a protection scheme analogous to that illustrated in FIG. 4A.

The preparation of a dual prodrug is illustrated in FIG. 4D. Compound 58 comprises two linking moieties 53 and 57 linked in reverse polarity by dual polarity linking moiety 55. Significantly, compound 58 has two free carboxyl groups for linking to the other moieties of the prodrug. Two molecules of biologically active entity 38 are coupled to the free carboxyl groups of compound 58 to yield dual prodrug 52. Cleavage of dual prodrug 52 in the vicinity of target cells liberates two molecules of released biologically active entity 39. Both molecules of released biological entity 39 comprise biologically active agent 38 derivatized with a leucine residue.

If the linking moiety is a peptide and the biologically active entity is a polypeptide and a terminus of the linking moiety is linked to a complementary terminus of the biologically active entity via an amide bond, the prodrug, or a portion thereof, can conveniently be prepared by recombinant synthesis. A nucleic acid coding for the amino acid sequence of the linking moiety and the biologically active agent can be prepared and used to express the covalent linking moiety—biologically active agent complex by standard techniques (see, e.g., Ausubel et al., 1987, Current Protocols in Molecular Biology, John Wiley & Sons, Inc., New York). The masking moiety can then be linked, for instance, to the amino terminus of the linking moiety by standard solution phase peptide chemistry. If the masking moiety is also a peptide or polypeptide and a terminus of the masking moiety is also linked to a complementary terminus of the linking moiety via an amide bond, the entire prodrug can conveniently be prepared by recombinant synthetic techniques. The nucleic acid expressing the prodrug should encode the amino acid sequences of the masking moiety, the linking moiety and the biologically active entity in tandem. Prodrugs produced by recombinant synthesis can be expressed in any eukaryotic or prokaryotic system in which the linking moiety is not cleaved by proteases, peptidases or other factors.

5.9 Formulation, Administration and Dosages

5.9.1 Compositions and Administration

The prodrugs of the invention can be used in a wide variety of applications to inhibit or prevent the growth of a tumor or target cell. For example, the prodrugs can be used to treat or prevent diseases related to tumor cell growth in humans and animals.

When used to treat or prevent cancer or diseases related thereto, the prodrugs of the invention can be administered or applied singly, as mixtures of prodrugs, in combination with other antineoplastic entities or in combination with other pharmaceutically active entities. A prodrug can be administered as the prodrug per se or may be in admixture with a variety of carriers, diluents or excipients as are well known in the art.

Pharmaceutical compositions comprising the prodrugs of the invention may be manufactured by means of conventional mixing, dissolving, levigating, emulsifying, encapsulating, ntrapping or lyophilizing processes. Pharmaceutical compositions may be formulated in conventional manner using one or more physiologically acceptable carriers, diluents, excipients or auxiliaries which facilitate processing of the active peptides into preparations which can be used pharmaceutically. Proper formulation is dependent upon the route of administration chosen.

Systemic formulations include those designed for administration by injection, e.g. subcutaneous, intravenous, intramuscular, intrathecal or intraperitoneal injection.

For injection, the prodrugs of the invention may be formulated in aqueous solutions, preferably in physiologically compatible buffers such as Hanks's solution, Ringer's solution, or physiological saline buffer. The solution may contain formulatory entities such as suspending, stabilizing and/or dispersing entities.

Alternatively, the prodrug may be in powder form for constitution with a suitable vehicle, e.g., sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic entity. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the prodrugs of the invention may contain charged side chains, they may be included in any of the above-described formulations as the free bases or as pharmaceutically acceptable salts. Pharmaceutically acceptable salts are those salts which substantially retain the activity of the free bases and which are prepared by reaction with inorganic acids. Pharmaceutical salts tend to be more soluble in aqueous and other protic solvents than are the corresponding free base forms.

5.9.2 Eff ctiv Dosages

The prodrugs of the invention, or compositions thereof, will generally be used in an amount effective to achieve the Intended purpose. Of course, it is to be understood that the amount used will depend on the particular application.

For example, for use as a antineoplastic entity, an therapeutically effective amount of a prodrug, or composition thereof, is applied or administered to an animal or human in need thereof. By therapeutically effective amount is meant an amount of peptide or composition that inhibits the growth of, or is lethal to, a target cell. The actual therapeutically effective amount will depend on a particular application. An ordinarily skilled artisan will be able to determine therapeutically effective amounts of particular prodrugs for particular applications without undue experimentation using, for example, the in vitro assays provided in the examples.

For use to treat or prevent tumor or target cell growth or diseases related thereto, the prodrugs of the invention, or compositions thereof, are administered or applied in a therapeutically effective amount. By therapeutically effective amount is meant an amount effective to ameliorate the symptoms of, or ameliorate, treat or prevent tumor or target cell growth or diseases related thereto. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating prodrug concentration range that includes the 150 as determined in cell culture (i.e., the concentration of test compound that is lethal to 50% of a cell culture), the MIC, as determined in cell culture (i.e., the minimal inhibitory concentration for growth) or the I₁₀₀ as determined in cell culture (i.e., the concentration of peptide that is lethal to 100% of a cell culture). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

The amount of prodrug administered will, of course, be dependent on the subject being treated, on the subject's weight, the severity of the affliction, the manner of administration and the judgment of the prescribing physician.

The antitumoral therapy may be repeated intermittently. The therapy may be provided alone or in combination with other drugs, such as for example other antineoplastic entities or other pharmaceutically effective entities.

5.9.3 Toxicity

Preferably, a therapeutically effective dose of the prodrugs described herein will provide therapeutic benefit without causing substantial toxicity.

Toxicity of the prodrugs described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the prodrugs described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingi et al., 1975, In: The Pharmacological Basis of Therapeutics, Ch.1, p.1).

### 6. EXAMPLE 1

### Tumor Selective Prodrug of TNFα

In this example, we describe a prodrug formulation of TNFα. To prepare the prodrug, the biologically active entity TNFα is linked to a plurality of polyethylene glycol masking moieties via tetrapeptide linking moieties.

The PEG moieties are linked to TNFα via the tetrapeptide linking moiety Ala-Leu-Ala-Leu. The tetrapeptide linker Ala-Leu-Ala-Leu is selected because it is known to allow the generation of protein-drug conjugates that are resistant to blood peptidases (Trouet et al., 1982, Proc. Natl. Acad. Sci. USA 79:626-629).

Leucyl-Derivatives of TNFα

With a prodrug comprising an alanyl-leucyl-alanyl-leucyl linker, extracellular hydrolysis in the tumor environment liberates a leucyl-derivative of the biologically active entity. To determine the appropriate stoichiometry for modification of TNFα, leucyl-derivatives are first prepared. Leucine residues are linked covalently through peptide bonds between their carboxyl group and a terminal amino group or side chain amino group (e.g. lysine ε-amine) of TNFα. The extent of modification is controlled by varying the ratio of reactive leucine molecules to TNFα.

The leucyl-derivatives of TNFα are prepared by acylation of free amino groups using the N-hydroxysuccinimide ester of acetol-leucine in an organic-aqueous medium. Hydroxyalkylated-amino acid-activated esters have excellent reactivity and are particularly well adapted to the acylation of proteins (Hermanson, 1996, Bioconjugate Techniques. Academic Press, San Diego, N.Y.; Geoghegan et al., 1979, Biochemistry 18:5392-5399). After deprotection of acetol-leucyl residues in mild conditions with NaIO₄, derivatives of TNFα are purified by size exclusion chromatography. Their purity is assessed by gel electrophoresis.

The activities of TNFα and of the leucyl-derivatives of TNFα are compared in order to select the extent of modification that is used for the preparation of prodrugs. A ratio leucine to PEG that preserves a significant amount of TNFα activity is selected as a basis for the TNFα prodrugs discussed below.

The activity of TNFα and its derivatives is determined using the classical assay as described (Creasey et al., 1987, Cancer Res. 47:145-149). L929 murine fibroblasts are seeded and grown in 96-well microtiter plates. They are then exposed to actinomycin D (1 μg/ml) in the presence of increasing concentrations of TNFα or Leuₓ-TNFα. After 24 hours of incubation at 37° C., cell lysis (lactate dehydrogenase [LDH] release assay) and viability (transformation of WST-1 by viable cells in a soluble formazan salt) are determined.

Receptor binding assays are also performed using HEp-2 cells (human epidermoid carcinoma cell line expressing mainly the 55 kDa TNFα receptor) to determine how the modifications influence receptor binding. Cells are exposed to [¹²⁵1]-TNFα and increasing concentrations of TNFα or Leuₓ-TNFα at 4° C. After several washes cell-associated radioactivity is quantified. HL60 cells (human promyelocytic leukemia cell line expressing mainly the 75 kDa receptor) are used similarly (Kuroda et al., 1995, Int. J. Cancer 63:152-157).

TNFα Prodrugs

TNFα is conjugated to one or more PEG molecules via the selected linker tetrapeptide attached through the C-terminal leucine of the tetrapeptide to primary amino groups of TNFα. The PEG-linker moieties are conjugated to a TNFα at an appropriate ratio as determined by the activity assays of the leucyl derivatives of TNFα discussed above. For instance, if leucyl-TNFα conjugates can tolerate modification of up to 56% of its amino groups while retaining significant activity, then the PEG-linker moieties are linked to TNFα at a molar ratio sufficient to modify 56% of the amino groups of TNFα. The optimum molecular weight of the PEG [H(OCH₂—CH₂)ₙ—OH] molecule is the lowest molecular weight that results in inactivation of the drugs with the conjugation level previously determined. PEG of molecular weight 1000, 4000, 8000, 10000 is available under its dialcohol form, while an ether form [H(OCH₂—CH₂)ₙOMe] with a molecular weight of 5000 can also be found.

The synthesis of the amino-protected tetrapeptide R¹-Ala-Leu-Ala-Leu can be performed using a classical solid phase peptide synthesis approach (Merrifield, 1963, JACS 85:2149-2154). Different amino-protecting groups (R¹) can be used depending on the expected utilization of the peptide. If necessary, the protected tetrapeptide can be purified by semi-preparative HPLC.

PEG can easily be coupled to a primary amine through a spacer or adaptor such as a succinate moiety. Succinylated PEG can be obtained commercially. Alternatively, well described and documented synthesis procedures are available from the literature (U.S. Pat. No. 5,612,460; U.S. Pat. No. 5,808,096). The PEGylated compounds can be purified by size exclusion chromatography.

Stability and Activity of TNFα Prodrug

Stability in blood and reactivation by tumor-secreted peptidases can be rapidly assessed in vitro. First, receptor-binding assays are carried out with the conjugates of TNFα to check inactivation as described, supra. Then, culture media conditioned by MCF-7/6 (breast carcinoma), LS-174-T (colon carcinoma), LNCaP (prostate carcinoma) and NCI-H209 (small-cell lung carcinoma), or other cell lines are used to check the reactivation of the conjugates by tumor-released peptidases. After incubation at 37° C. for increasing time lags, the conjugates are analyzed by western immunoblotting or by an activity assay.

The prodrugs are then tested in vivo (acute toxicity/lethality studies and chemotherapeutic activity), both as single entities and in combination with other cytotoxic and/or cytostatic anticancer entities such as doxorubicin. OF-1 mice are used for lethality studies with the conjugates. Increasing doses of the conjugates or of the parent compound are administered by the iv. route, and the LD₅₀ values are determined after 28 days. Single as well as multiple injection protocols are considered.

For the chemotherapeutic evaluation of the conjugates, Balb/c nu-nu mice are implanted subcutaneously in both flanks with fragments of human tumors grown from the cell lines previously mentioned. Tumors are allowed to grow until they reach a mean diameter of at least 6 mm. Then, treatments consisting of saline, doxorubicin, the prodrug alone or the prodrug in combination with doxorubicin, are administered as i.v. bolus injections. The animals are treated once a week for five consecutive weeks. Clinical signs, body weight and tumor growth are monitored for at least 60 days. Treatment efficacy is assessed based on tumor growth delays and on the ratio of tumor volumes in treated groups versus control animals.

### 7. EXAMPLE 2

### Tumor-Activated Dual TNFα—Doxorubicin Prodrug

In this example, we present a dual prodrug that releases TNFα and the antineoplastic entity doxorubicin at target cells in vivo.

First, -Mal-Leu-OH derivatives of TNFα are prepared. The amino terminus of leucine methyl ester (Leu-OMe) is modified with dimethylmaleic anhydride to yield dimethylmaleyl leucine (Mal-Leu-OMe). Free amino moieties of TNFα are then modified by forming amide bonds between free amino groups of TNFα and free carboxyl groups of -Mal-Leu-OMe. After enzymatic ester hydrolysis (Shin C. G., 1997, Bull. Chem. Soc. Jpn. 70, 1427-1434) of the Leu residues, the resulting -Mal-LeuOH TNFα derivatives are compared to native TNFα in terms of activity. The maximum number of free amino moieties of TNFα that can be modified with -Mal-LeuOH without significantly altering the activity is determined as discussed in Example 1, supra.

Using the determined stoichiometry, TNFα is similarly modified with Mal-Leu-Ala-Leu-Ala-Leu-Dox in order to obtain the dual prodrug. The peptide-Dox conjugate is prepared according to U.S. Pat. No. 5,962,216.

Alternative dual prodrugs are also prepared by coupling leucine directly to the free carboxyl groups available in TNFα. TNFα has 5 aspartic acid and 9 glutamic acid residues.

The TNFα derivatives are purified by size-exclusion chromatography, and the purity is determined by electrophoretic techniques.

The activity and/or inactivity, stability and reactivation of the dual prodrugs is assayed as described in Example 1, supra. In vivo toxicity and activity of the dual prodrugs is also evaluated as described in Example 1, supra.

### 8. EXAMPLE 3

### Tumor-Activated IGF-1 Antagonist Prodrug

In this example, we demonstrate a prodrug comprising an oligopeptide antagonist of insulin-like growth factor-1 (IGF-1) linked to PEG via a tetrapeptide linking moiety.

The selected IGF-1 antagonist is a cyclic dodecapeptide made of D-amino acids. It has the formula cyclo[H-D-Cys-D-Ser-D-Lys-D-Ala-D-Pro-D-Lys-D-Leu-D-Pro-D-Ala-D-Ala-D-Tyr-D-Cys-OH]. The peptide is cyclized via a disulfide bridge between the side chains of the two cysteine residues. It is synthesized by standard solid phase peptide synthesis techniques.

Leucyl Conjugates of the IGF-I Antagonist

Free amino groups of the IGF-1 antagonist are modified with leucine residues as described in Example 1, supra. Initially, only the terminal amino group of the IGF-1 antagonist is modified. If modification of the terminal amino group results in a significant loss of activity, then other reactive groups of IGF-1 are modified and assayed for retention of functional activity.

The conjugates are purified by semi-preparative reverse phase HPLC. The purity of the conjugates is determined by reverse phase HPLC-MS. The structural quality of the conjugates is verified by NMR and mass spectrometry.

The activities of the IGF-1 antagonist and its leucyl-derivative are compared in receptor binding and cell proliferation assays. MCF-7/6-human breast cancer cells are seeded and grown in serum-free medium. They are then incubated at 4° C. with [¹²⁵I]-IGF-1 in the presence of increasing concentrations of IGF-1, the IGF-1 antagonist or its leucyl-derivative. After washes, cell-associated radioactivity is quantified.

MCF-7/6 cells are also used to compare the inhibitory effects of the IGF-1 antagonist and of its leucyl-derivative on IGF-1-induced proliferation. Cells are seeded in serum-free medium in the presence of 10 ng/ml IGF-1 and increasing concentrations of the IGF-1 antagonist or its leucyl-derivative. Cell proliferation is then estimated by the incorporation of [³H]-thymidine into DNA.

IGF-1 Antagonist Prodrug

The IGF-1 antagonist is conjugated to one or more PEG molecules via the selected linker tetrapeptide attached through the C-terminal leucine of the tetrapeptide to primary amino groups the antagonist as described in Example 1.

Radiolabeled (¹⁴C or ³H) amino acids are incorporated in a fraction of the prodrugs. The resulting radioactive conjugates are then used as tracers to allow sensitive detection of metabolites in the in vitro studies. All PEGylated compounds are purified by size exclusion chromatography.

The inactivation of the IGF-1 prodrug is tested using the activity assays described above for the leucyl derivatives of IGF-1. Blood stability and reactivation in tumor cells conditioned media is tested as described in Example 1 and additionally by HPLC analysis. Toxicity and chemotherapeutic activity studies are then performed with the prodrug and with the prodrug in combination with doxorubicin. These studies are once again performed as described in Example 1 and additionally by HPLC.

### 8. EXAMPLE 4

### Tumor-Activated Lytic Peptide Prodrug

In this example, we describe a prodrug comprising a lytic peptide linked to a PEG masking moiety via a tetrapeptide linking moiety.

Synthesis of the Lytic Peptide LK15 C:

The lytic peptide is composed exclusively of leucine and lysine residues, like those described as cytolytic in the literature (Castano et al., 1999, supra). However, only D-amino acids are used to yield a relatively low sensitivity to proteolysis in vivo. A 15-mer (LK15) containing 10 D-leucine and 5 D-lysine residues with the structure H-D-Lys-D-Leu-D-Leu-D-Lys-D-Leu-D-Leu-D-Leu-D-Lys-D-Leu-D-Leu-D-Leu-D-Lys-D-Leu-D-Leu-D-Lys-OH is prepared according to standard solid phase peptide synthesis techniques using orthogonal protecting groups on the E-amino groups of lysine side chains as necessary.

Leucyl Conjugates of the Lytic Peptide

Free amino groups of the lytic peptide are modified with leucine residues as described in Example 1, supra. Initially, the E-amino group of each lysine side chain of the lytic peptide is modified. If necessary, the terminal amino group is also modified with leucine. The orthogonal protecting groups of the synthesis step are exploited to selectively modify specific amino groups.

The conjugates are purified by semi-preparative reverse phase HPLC. The purity of the conjugates is determined by reverse phase HPLC-MS. The structural quality of the conjugates is verified by NMR and mass spectrometry.

The activity of the lytic peptide and its derivatives are assessed in hemolytic assays as well as by quantifying LDH release from tumor cells. Erythrocytes are isolated from fresh human blood collected on citrate from healthy donors. Peptide dilutions are dispensed in 96-well plates before the addition of an erythrocyte suspension to each well. After a 30-minute incubation at 37° C. and a centrifugation, the supernatants are transferred to new plates for A₄₁₄ determination. Blank (no hemolysis) values are obtained from unexposed cells and 100% hemolysis is determined from cells suspended in distilled water. To check if serum alters the lytic activity of the peptide by potential alterations of the monomer-polymer equilibrium, the assay is also performed with various concentrations of human serum (10 to 100%).

The lytic activity is also determined on nucleated cancer cells. We assess lysis based on the extent of LDH release from cells exposed to increasing concentrations of LK15 or its leucyl-derivatives. MCF-7/6 cells are grown in 96-well plates and then exposed to increasing concentrations of the compounds to be tested for 1 hour at 37° C. Supernatants and the cell monolayers are separated and used for the determination of LDH activity. The percentage of total activity released in the culture medium is considered.

Lytic Peptide Prodrug

The lytic peptide is conjugated to one or more PEG molecules via the selected linker tetrapeptide attached through the C-terminal leucine of the tetrapeptide to primary amino groups of the lytic peptide as described in Example 1. The choice of primary amino groups and stoichiometry is determined by the activity results from the leucyl derivatives of the lytic peptide as discussed for TNFα in Example 1.

The inactivation of the lytic peptide prodrug is tested using the activity assays described above for the leucyl derivatives of lytic peptide. Blood stability and reactivation in tumor cells conditioned media is tested as described in Example 1 and additionally by HPLC analysis. Toxicity and chemotherapeutic activity studies are then performed with the prodrug and with the prodrug in combination doxorubicin. These studies are once again performed as described in Example 1 and additionally by HPLC.

### 9. EXAMPLE 5

### Tumor-Activated Dual Antiangiogenic Peptide—Doxorubicin Prodrugs

In this example, we describe a dual prodrug that releases doxorubicin and an antiangiogenic peptide at or near tumors in vivo.

Reverse sequences made of D-amino acids have been developed, derived from the second type-1 repeat of thrombospondin-1 (TSP-1). These peptides have antiproliferative and antiangiogenic properties (Dawson et al, 1999, supra). Mal II, a 19-residue peptide is derived from the properdin-like repeat of TSP-1 and shown to possess potent antiangiogenic properties when any one of three L-amino acids are substituted by their D-enantiomers. In vitro and in vivo anti-angiogenic activities are achieved by low concentrations. The most interesting such peptide is D-Ile¹⁵-Mal II (Dawson et al., 1999, supra). It is quite amenable to alterations since it can be shortened to as little as seven amino acids with no loss of activity, and the addition of an ethylamide end group to the 7-mer further increases its potency.

A heptapeptide derivative of D-Ile¹⁵-Mal II with the structure Acetyl-Gly-Val-D-Ile-Thr-Arg-Ile-Arg is synthesized by standard solid phase peptide synthesis techniques. Also synthesized via standard techniques are an ethylamide-capped derivative of the heptaptide with the structure Acetyl-Gly-Val-D-Ile-Thr-Arg-Ile-Arg-NHEt and the D-heptapeptide D-Arg-D-Ile-D-Arg-D-Thr-D-Ile-D-Val-Gly derived from the heptapeptide.

The heptapeptide and the two derivatives are each first coupled to a leucine residue. For the capped heptapeptide, the leucine residue is coupled to the side chain of an Arg residue via a dimethylmaleyl dicarboxylic adaptor according to standard techniques. For both other peptides, the leucine residue is linked through its amino group to the carboxy terminus. The activity of the resulting peptides are compared to the activities of the corresponding native peptides. The activities of the compounds are assessed on endothelial cells such as the EAhy926 cell line (Edgell et al., 1983, Proc. Natl. Acad. Sci. USA 80:3734-3737). After incubation with various concentrations of the compounds in the presence of vascular endothelium growth factor (VEGF), cell viability (WST-1 reagent) is determined as well as the effect on [³H]-thymidine incorporation into DNA.

The three native heptapeptides are then each conjugated via standard synthetic techniques to Leu-Ala-Leu-Ala-Leu-Dox to produce the dual prodrugs. For the capped heptapeptide, the Leu-Ala-Leu-Ala-Leu-Dox construct is coupled to the side chain of an Arg residue via a dimethylmaleyl dicarboxylic spacer according to standard techniques. The quality of all compounds are determined by reverse phase HPLC(-MS). When necessary, semi-preparative HPLC is used for purification. The structures are confirmed by amino acid, NMR and MS analyses.

For each dual prodrug, its stability in whole human blood as well as its reactivation by tumor cells conditioned media are evaluated. The stability in blood and reactivation by conditioned media are studied as described above using HPLC and radiolabeled molecules (a dual prodrug containing a labeled amino acid in the antiangiogenic peptide portion is used as a tracer) to allow the detection of the angiogenic peptide and its derivatives. Fluorescence detection is used for the anthracycline and its derivatives.

For each dual prodrug, its in vivo toxicity is then evaluated as described in Example 1, supra. Its chemotherapeutic activity is then compared to that of Dox and of the corresponding native antiangiogenic peptide, alone or in combination.

### 10. EXAMPLE 6

### Tumor-Activated Dual IGF-1 Antagonist—Doxorubicin Prodrug

In this example we describe a dual prodrug that releases doxorubicin and an IGF-1 antagonist at or near a tumor in vivo.

The IGF-1 antagonist described in Example 3, supra, is used to prepare a dual prodrug. For the dual prodrug, conjugation takes place at the carboxy-terminus of the antagonist rather than on its free N-terminal amino group.

Ideally, adding a tumor peptidase-sensitive peptide derivative of Dox to the carboxyl group of the relatively small antagonist prevents it from binding the IGF-1 receptor (through a steric hindrance phenomenon). If so, the dual conjugate is inactive, with no requirement for a further masking of the extracellularly-acting antagonist. If masking is required, the two lysine residues of the antagonist are modified with pH-sensitive, e.g. dimethylmaleyl, groups that are removed in the tumor environment.

A leucyl derivative of the IGF-1 antagonist is prepared by standard coupling at the C-terminus of the peptide utilizing suitable orthogonal protecting groups. If necessary, the disulfide bridge is recylized by standard techniques. The resulting C-terminal leucyl derivative of the IGF-1 antagonist is then coupled at its C-terminus with the N-terminus of Ala-Leu-Ala-Leu-Dox by standard techniques. It is believed that the resulting dual prodrug, when in the tumor microenvironment, yields the leucyl-derivatives of both Dox and the IGF-1 antagonist.

The leucyl-derivative and the prodrug are purified by semi-preparative reverse phase HPLC. The purity of the conjugates is determined by reverse phase HPLC-MS. The structural quality of the conjugates is verified by NMR and mass spectrometry.

First, the activity of the leucyl-derivative is assayed as described in Example 3. Second, the inability of the full prodrug to bind IGF-1 receptors is assayed. Its blood stability and reactivation by tumor cells conditioned media are then assayed. Fluorescence detection is used to detect the anthracycline and its derivatives. If the activity of the IGF-1 antagonist is not inhibited in the dual prodrug, the ε-amino groups of the 2 lysine residues are masked, for example with pH sensitive moieties such as dimethylmaleyl groups.

Once again, after the in vitro tests, the in vivo toxicity of the dual prodrug is evaluated, and its chemotherapeutic activity is compared to that of Dox and of the IGF-1 antagonist, alone or in combination. Nude mice bearing MCF-7/6 human breast tumors are used for the activity assays.

### 11. EXAMPLE 7

### Tumor-Activated Dual Lytic Peptide—Doxorubicin Prodrug

In this example, we present a dual prodrug that specifically releases a lytic peptide and doxorubicin at or near target cells in vivo.

A dual prodrug is also prepared with the lytic peptide described in Example 4, supra. The optimal site of conjugation is as determined in Example 4. For instance, the free carboxy terminus is a likely effective site of conjugation. The lytic activity of the lytic peptide is reversibly masked as described in Example 4.

The LK15 peptide is be conjugated to Leu-Ala-Leu-Ala-Leu-Dox by standard synthetic techniques via a dimethylmaleyl dicarboxylic adaptor.

Analytical characterization, activity assays, blood stability assays, reactivation assays, and lethality assays are performed as described in Example 4, supra. Chemotherapy studies as described in Example 4, supra, and studies with colo-rectal tumors that are relatively resistant to anthracyclines are also performed (e.g. LS-174-T). The results are compared to the naked lytic peptide, to doxorubicin and to the combination of the two entities.

### 12. EXAMPLE 8

### Tumor Activated Dual Substance P Antagonist—Doxorubicin Prodrug

In this example, we describe dual prodrugs that selectively release a substance P antagonist and doxorubicin at or near a tumor in vivo.

One of the most potent substance P antagonists, exhibiting the broadest spectrum of activity, is the 11-residue amidated peptide D-Arg-Pro-Lys-Pro-D-Trp-Gln-D-Trp-Phe-D-Trp-Leu-Leu-NH₂ (“SPD”). This peptide showed activity in vivo when injected Intratumorally, peritumorally, or Lp. (Secki et al., 1997, supra).

SPD has no free carboxyl group to use to couple the linker peptide-Dox conjugate, and the amidated carboxy terminal Leu is very likely important for activity. Therefore, the N-terminus of SPD (D-Arg) is modified with a dicarboxylic methylmaleyl moiety according to a previously described synthesis procedure using classical solution peptide chemistry with standard orthogonal side chain protecting groups (Nyeki, 1998, J. Peptide Sc. 4:486-495).

The amino terminus of a leucine residue is then coupled to the free carboxyl group introduced on the amino-terminal D-arginine. The leucine derivative is then coupled to the free terminal amino-group of Ala-Leu-Ala-Leu-Dox.

Reactivation by tumor-released peptidases is believed to yield leucyl-Dox and leucyl-dimethylmaleyl-SPD or SPD. Leucyl-dimethylmaleyl-SPD is therefore also synthesized and tested for its activity or further activation.

The purity of the dual prodrug and its derivatives is determined by HPLC analysis. If necessary, semi-preparative HPLC is used for purification. The structures of the dual prodrug and its derivatives are checked by amino acid analysis, NMR and MS analyses.

First the ability of the dual prodrug to inhibit the binding of [¹²⁵I]-Bradykinin to Swiss-3T3 cells is assayed. If the dual prodrug is unexpectedly active as an inhibitor, its activity is masked, for example by reversibly modifying the side chain amino groups as described in Example 3.

Then, blood stability and reactivation by tumor cell conditioned media are assayed. Fluorescence and UV detection allow detection of doxorubicin and the substance P antagonist The activity of the metabolites generated in conditioned media is assayed on human lung cell lines (COR-L23 or NCI-H69 for example). Cytotoxicity and proliferation assays are performed as described in Example 1 and in Example 3.

Lethality studies are then performed followed by experimental chemotherapy of different human lung tumors implanted in nude mice as described in Example 1.

### 13. EXAMPLE 9

### Tumor-Selective Transport Peptide (e.g. Tat) for Intracellular Delivery of Doxorubicin

In this example we describe the design and preparation of an HIV Tat-derived transport peptide (Gly-Arg-Lys-Lys-Arg-Arg-Gln-Arg-Arg-Arg-Pro-Pro-Gln-Cys) prodrug for carrying doxorubicin directly to the nucleus of a target cell. Formulation of the construct as a prodrug according to the present invention by coupling with PEG-Ala-Leu-Ala-Leu provides selectivity for target cells to the construct and increases its stability. The HIV-Tat-derived transport peptide of the construct carries doxorubicin to the nucleus of the cell, doxorubicin's site of action.

Leucyl-Derivatives of the Transport Peptide

Since selective cleavage of the linking tetrapeptide likely liberates a leucyl-derivative of the HIV-Tat-derived transport peptide construct, leucyl derivatives of the HIV-Tat-derived transport peptide are first synthesized to identify those leucyl derivatives that retain their ability to enter cells.

Leucine residues are linked covalently through peptide bonds between their carboxyl group and a side chain amino (e.g. lysine residues) or guanidino (e.g. arginine residues) group present or added to the HIV-Tat-derived transport peptide. Derivatives are then compared with regard to their capacity to be internalized.

Alternatively, transport activity can be reversibly blocked by capping the side chains of lysine or arginine residues with acid-labile moieties such as dimethylmaleyl or citraconyl groups. These groups are introduced from the corresponding anhydrides and are unstable when the pH falls below 6.5, values often encountered in the vicinity of tumor cells (Lavie et al., 1991, supra).

The purity of each synthesized compound is determined by reverse phase HPLC. If necessary, compounds are purified by semi-preparative or preparative HPLC, and structural information is obtained from amino acid analysis, MS, NMR and elemental analyses.

The uptake of the biotinylated peptide and its leucyl-derivatives in MCF-7/6 human breast cancer cells is determined after reaction with streptavidin-conjugated horse radish peroxidase. Cells are incubated for 1 to 18 hours with biotinylated peptides at 0.1 to 20 μg/ml, washed, fixed with ethanol, permeabilized with Triton X-100 and incubated with a streptavidin-peroxidase conjugate (5 μg/ml) for 1 hour. After more washes, the peroxidase activity is quantified using o-phenylenediamine and H₂O₂ as the substrates.

PEG-Ala-Leu-Ala-Leu Derivatives of the Transport Peptide

Once the best position for conjugation is determined, PEG-Ala-Leu-Ala-Leu is introduced at that position as described in Example 1, supra If several leucyl-derivatives display adequate transport activity, then several PEG-Ala-Leu-Ala-Leu conjugates are prepared. The ideal PEG molecular weight to prevent cell penetration is determined by the cellular uptake assays described above.

PEG-linker tetrapeptide conjugates of the HIV-Tat-derived transport peptide are tested similarly for cell uptake. In addition, their blood stability and reactivation by cancer cells conditioned media is also be assessed. Conjugates incorporating radiolabeled residues are used as tracers in these studies to allow sensitive detection of the conjugates and of potential metabolites. The peptide conjugates are incubated at 37° C. in whole blood from healthy donors collected in citrated tubes. At selected time points, samples are centrifuged to eliminate cells and analyzed by HPLC. The disappearance of the conjugates over time is quantified. Media conditioned by MCF-7/6 (breast carcinoma) and LS-174-T (colon carcinoma) cell lines are used to check tumor peptidase reactivation of the conjugates. After incubation at 37° C. for increasing time lags, the conjugates and their metabolites are analyzed by HPLC.

In vivo tissue distribution studies are then performed to confirm the selective delivery to tumors. Balb/c nu-nu mice are implanted subcutaneously in both flanks with fragments of human tumors that will be allowed to grow until they reach a mean diameter of at least 6 mm. Treatments consisting of a biotinylated and/or radiolabeled peptide conjugate, are administered as i.v. bolus injections. At selected time points, tumors, heart, liver, kidneys, spleen, brain, lungs, and plasma are collected. After tissue homogenization, the biotinylated and/or radiolabeled peptide is isolated on steptavidin-coated multiwell plates and quantified.

Doxorubicin Prodrugs

Doxorubicin conjugates of the tumor-selective vector peptide are then prepared by coupling to the appropriate PEG-Ala-Leu-Ala-Leu derivatives of the HIV-Tat-derived transport peptide. The carboxy terminus of the transport peptide is coupled to the free hydroxyl group of doxorubicin via standard synthesis techniques. In a second prodrug, a glutaric anhydride spacer is linked to the free hydroxyl moiety of doxorubicin via an ester linkage and to the amino terminus of the transport peptide.

A third construct is prepared by coupling doxorubicin to the transport peptide via the daunosamine moiety of doxorubicin through an amide linkage. In this case, a peptide spacer that is cleaved intracellularly (in the Golgi apparatus or in the nucleus) to liberate free doxorubicin is used between the drug and the carrier peptide. A suitable spacer is identified be screening peptide spacers in the presence of tumor cell conditioned media on one hand or in the presence of tumor cell homogenates (or subcellular fractions). The peptidic spacer is selected on the basis of its resistance to the conditioned media and its sensitivity to the cell homogenates at neutral pH.

The stability of the doxorubicin-carrier peptide conjugate in whole human blood is assayed as described in Example 1, supra, prior to evaluation of its in vivo toxicity (lethality studies) and chemotherapeutic activity In nude mice bearing subcutaneous resistant tumors such as the human breast cancer MCF-7/Adr.

### 14. EXAMPLE 10

### Pro-Apoptopic Protein-Carrier Peptide Prodrug

In this example, we describe a prodrug that selectively delivers a pro-apoptotic protein construct to target cells. The construct includes a transport peptide that carries the pro-apoptotic protein into the nucleus of the target cell and the pro-apoptotic protein granzyme B.

Granzyme B—Transport Peptide Prodrug

Granzyme B is purified to homogeneity from the YT natural killer cell line (Harris et al., 1998, J. Biol. Chem. 273:27364-27373). Alternatively, the recombinant enzyme is expressed and purified from the yeast Pichia pastoris (Sun et al., 1999, Biochem. Biophys. Res. Commun. 261:251-255).

The construct of granzyme B and the transport peptide is prepared by standard recombinant techniques. The amino terminus of the transport peptide is fused to the carboxy terminus of granzyme B. The conjugate is tested in vitro for its apoptosis-inducing properties on different tumor cell types. Pro-caspase 3 activation assays as well as DNA fragmentation are used to check its activity (Sun et al., 1999, supra).

If the resulting construct allows the intracellular incorporation and pro-apoptotic action of granzyme B, a tumor-specific prodrug formulation of the construct is prepared as described in Example 9, supra. A PEG-Leu-Ala-Leu-Ala-Leu derivative of the transport peptide is first prepared and then conjugated to granzyme B. Alternatively, a Leu-Ala-Leu-Ala-Leu-PEG derivative of the transport peptide is prepared and then conjugated to granzyme B.

The stability in whole human blood and the reactivation by tumor cells conditioned media of the full tumor-specific conjugate is analyzed by HPLC using radiolabeled conjugates as described in Example 1, supra. In vivo evaluation then includes toxicity studies in normal mice, and experimental chemotherapy of human tumor xenografts in nude mice as described in Example 1, supra.

Doxorubicin—Granzyme B Prodrugs

The pro-apoptopic effect of the granzyme B prodrug could be reinforced by the administration of doxorubicin. This could also be a way to overcome resistance to doxorubicin, which often is associated with a defect in the induction of apoptosis (Dive, 1997, J. Int. Med. 242:139-145; Haq and Zanke, 1998, Cancer Metast. Rev. 17, 233-239; Dennis and Kastan M. B., 1998, Drug Resistance Updates 1:301-309, 1998).

As a first approach, the granzyme B prodrug is administered with any of the doxorubicin prodrugs described above. Additionally, a doxorubicin prodrug is prepared comprising the PEG-Ala-Leu-Ala-Leu derivative of the transport peptide. In this prodrug, doxorubicin is conjugated to the transport peptide by a peptide spacer that is capable of being specifically cleaved by the serine protease granzyme B within a cell. The spacer is developed based on the known specificity of granzyme B. For optimal therapeutic effect, the two prodrugs are administered sequentially to allow intratumoral accumulation of granzyme B prior to uptake of the granzyme B-sensitive doxorubicin construct.

As a third approach, a dual prodrug formulation of doxorubicin and the granzyme B—transport peptide construct is prepared. In this dual prodrug, doxorubicin is linked to the transport peptide construct via an ester or a peptide spacer that remains stable in tumor conditioned media as well as in the presence of granzyme B, but is sensitive to other intracellular hydrolases.

The present invention is not to be limited in scope by the exemplified embodiments, which are intended as illustrations of single aspects of the invention, and any compositions and methods which are functionally equivalent are within the scope of the invention. Indeed, various modifications of the invention in addition to those described above will become apparent to those skilled in the art from the foregoing description and accompanying drawings. Such modifications are intended to fall within the scope of the appended claims.

All patents and publications cited herein are hereby incorporated by reference in their entirety.

1. A prodrug composition according to formula (I):
(M−L¹)ₙ−B (I)
wherein b is a biologically active entity comprising a polypeptide or an extracellularly active entity; each L¹ is independently a linking moiety; each M is independently a masking moiety such that (M−L¹)ₙ hinders the activity of B and is susceptible to cleavage at or near a tumor or a target cell; and n is an integer from 1 up to the total number of reactive groups of B.

2. The prodrug composition according to claim 1 in which each L¹ is the same.

3. The prodrug according to claims 1 and 2 in which each L¹ is independently a peptide comprising an amino acid sequence selected from the group consisting of (Leu)_{y}(Ala-Leu)ₓAla-Leu and (Leu)_{y}(Ala-Leu)ₓAla-Phe wherein y=0 or 1 and x=1, 2, or 3.

4. The prodrug according to claim 3 in which each L¹ is independently a peptide comprising the amino acid sequence Ala-Leu-Ala-Leu or the amino acid sequence Leu-Ala-Leu-Ala-Leu.

5. The prodrug according to any of claims 1 to 4 in which each (M−L¹) is covalently linked to an amino-terminus of B, to an amino acid side chain of B, to a lysine side chain of B or to an arginine side chain of B.

6. The prodrug according to any of claims 1 to 5 in which from about 36% up to about 86% of the free reactive groups of B are blocked with (M−L¹) groups.

7. The prodrug according to any of claims 1 to 6 in which M or L¹ includes an adaptor moiety.

8. The prodrug according to claim 7 in which the adaptor moiety is selected from the group consisting of citraconyl, dimethylmaleyl, succinyl, glutaryl and diglycolyl.

9. The prodrug according to any of claims 1 to 8 in which M or L¹ includes a spacer moiety.

10. A prodrug according to any of claims 1 to 9 in which M reduces or prevents the in vivo cleavage of (M−L¹) in normal tissues and body fluids.

11. The prodrug according to any of claims 1 to 10 in which M is a polymer.

12. The prodrug according to claim 11 in which M is a polyalkylene glycol.

13. The prodrug according to claim 12 in which M is a polyethylene glycol having an average molecular weight of from about 1000 Da up to about 12000 Da.

14. The prodrug according to any of claims 1 to 10 in which M is selected from the group consisting of a polypeptide, an immunoglobulin, an antibody and albumin.

15. The prodrug according to any of claims 1 to 10 in which M is selected from the group consisting of an N-terminally blocked amino acid and a genetically non-encoded amino acid.

16. The prodrug according to claim 15 in which M is a D-amino acid.

17. The prodrug according to claim 14 or 15 in which M is N-Me-alanine, D-alanine or β-alanine.

18. The prodrug according to any of claims 1 to 17 in which M is negatively charged at physiological pH.

19. The prodrug according to any of claims 1 to 18 in which M is a biologically active entity.

20. The prodrug according to claim 19 in which M is selected from the group consisting of anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecins, proteasome inhibitors, farnesyl-protein transferase inhibitors, epothilones, maytansinoids, discodermolide, platinum derivatives, duocarmycins, combretastatin, epipodophyllotoxins, TNFα, IFN-α, IFN-γ, IL-1, IL-2, IL-6, an IGF-1 antagonist, a lytic peptide, an anti-angiogenic peptide, a thrombospondin-derived peptide, a substance P antagonist, TRAIL (Apo-2 ligand) and Fas ligand.

21. The prodrug according to any of claims 1 to 20 in which (M−L¹)ₙ lowers the in vivo toxicity of B.

22. The prodrug according to any of claims 1 to 21 wherein B is selected from the group consisting of TNFα, IFN-α, IFN-γ, IL-1, IL-2, IL-6, an IGF-1 antagonist, a lytic peptide, an antiangiogenic peptide, a thrombospondin-derived peptide, a substance P antagonist, TRAIL (Apo-2 ligand) and Fas ligand.

23. The prodrug according to any of claims 1 to 21 in which B is a construct comprising a transport peptide and a biologically active entity.

24. The prodrug which is a composition according to formula (II):
(M−L¹)ₚ−TP−(L²−A)ₘ (II)
wherein M and L¹ are as defined in any of claims 1 to 21; each A is independently an intracellularly active biologically active entity; each L² is independently an optional linking moiety susceptible to cleavage within a cell; TP is a polypeptide capable of carrying (L²−A)ₘ into a cell; m is an integer from 1 up to (k−1) and p is an integer from 1 up to (k−m) wherein k is an integer equal to the total number of reactive groups of TP.

25. The prodrug according to claim 24 in which m is 1 and p is 1.

26. The prodrug according to claims 24 and 25 in which (M−L¹) is linked to one of the termini of TP and (L²−A) linked to the other terminus of TP.

27. The prodrug according to any of claims 24 to 26 in which (M−L¹)ₙ prevents TP from carrying A into a cell.

28. The prodrug according to any of claims 24 to 27 in which A is a drug, a polypeptide, a nucleic acid or an analog thereof, or a marker molecule.

29. The prodrug according to claim 28 wherein A is selected from the group consisting of anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecins, proteasome inhibitors, farnesyl-protein transferase inhibitors, epothilones, maytansinoids, discodermolide, platinum derivatives, duocarmycins, combretastatin, epipodophyllotoxins, BH3 peptides, p53 peptides, caspases, granzyme B, ribozymes, antisense oligonucleotides, c-DNAs, peptide nucleic acids, rhodamine, FITC, biotin and GFP.

30. The prodrug according to any of claims 24 to 29 in which TP is selected from the group consisting of Antennapedia homeodomain derived peptides, Tat transactivation protein derived peptides, arginine oligomers and peptides derived from the CDR region of polyreactive anti-DNA antibodies.

31. The prodrug according to claim 30 wherein TP comprises an amino acid sequence selected from the group consisting of

| | |
|---|---|
| KKWKMRRNQFWVKVQRG; | (SEQ ID NO:6) |
| GRKKRRQRRRPPQC; | (SEQ ID NO:7) |
| RRRRRRRRR; and | (SEQ ID NO:8) |
| VAYISRGGVSTYYSDTVKGRFTRQKYNKRA. | (SEQ ID NO:9) |

32. The prodrug according to any of claims 24 to 31 in which TP comprises one or more D-amino acids.

33. The prodrug according to claim 32 wherein TP comprises an amino acid sequence selected from the group consisting of

| | |
|---|---|
| kkwkmrrnqfwvkvqrg; | (SEQ ID NO:10) |
| grkkrrqrrrppqc; | (SEQ ID NO:11) |
| rrrrrrrrr; and | (SEQ ID NO:12) |
| vayisrggvstyysdtvkgrftrqkynkra. | (SEQ ID NO:13) |

34. The prodrug according to any of claims 24 to 33 in which L² is a peptide susceptible to cleavage by intracellular proteases.

35. The prodrug according to claim 34 in which L² is selected from the group consisting of caspase substrates, furin substrates, proteasome substrates and granzyme B substrates.

36. A dual prodrug composition according to formula (III):
(D¹−L³)ₙ−D² (III)
wherein D¹ and D² are each independently biologically active entities and L³ is a linking moiety susceptible to cleavage at or near a tumor or a target cell.

37. The dual prodrug according to claim 36 in which L³ comprises an adaptor moiety, said adaptor moiety comprising two carboxylic acid moieties or two amino moieties.

38. The dual prodrug according to claim 37 in which the adaptor moiety is selected from the group consisting of citraconyl, dimethylmaleyl, succinyl, glutaryl and diglycolyl.

39. The dual prodrug according to any of claims 36 to 38 in which n is 1.

40. The dual prodrug according to any of claims 36 to 39 in which D¹ and D² constitute a pair of biologically active molecules that act in concert.

41. The dual prodrug according to any of claims 36 to 40 in which D¹ or D² is an intracellularly active biologically active entity.

42. The dual prodrug according to any of claims 36 to 40 in which D¹ and D² are both intracellularly active biologically active entities.

43. The dual prodrug according to any of claims 36 to 40 in which D¹ or D² is an extracellularly active biologically active entity.

44. The dual prodrug according to any of claims 36 to 40 in which one of D¹ or D² is an intracellularly active biologically active entity and the other is an extracellularly active biologically active entity.

45. The dual prodrug according to any of claims 36 to 44 wherein D¹ or D² is selected from the group consisting of anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecins, proteasome inhibitors, farnesyl-protein transferase inhibitors, epothilones, maytansinoids, discodermolide, platinum derivatives, duocarmycins, combretastatin, epipodophyllotoxins, TNFα, IFN-α, IFN-γ, IL-1, IL-2, IL-6, an IGF-1 antagonist, a lytic peptide, an anti-angiogenic peptide, a thrombospondin-derived peptide, a substance P antagonist, TRAIL (Apo-2 ligand) and Fas ligand.

46. The dual prodrug according to claim 45 in which one of D¹ or D² is TNFα and the other of D¹ or D² is an antitumor entity.

47. The dual prodrug according to claim 46 in which one of D¹ or D² is TNFα and the other of D¹ or D² is an interferon, IFN-α or IFN-γ.

48. The dual prodrug according to claim 45 in which D¹ is doxorubicin and D² is selected from the group consisting of TNFα, IGF-1 antagonist, a lytic peptide, an antiangiogenic peptide, substance P antagonist, a proteasome inhibitor and a farnesyl-protein transferase inhibitor.

49. The dual prodrug according to any of claims 36 to 46 in which D¹ or D² is a construct comprising an intracellularly active biologically active entity, a polypeptide capable of carrying the intracellularly active biologically active entity into a cell and an optional linking moiety susceptible to cleavage within a cell.

50. The dual prodrug according to claim 49 in which D¹ and D² are each independently constructs comprising an intracellularly active biologically active entity, a polypeptide capable of carrying the intracellularly active biologically active entity into a cell and an optional linking moiety susceptible to cleavage within a cell.

51. The dual prodrug according to claim 49 or 50 in which the intracellularly active biologically active entity is selected from the group consisting of anthracyclines, doxorubicin, daunorubicin, folic acid derivatives, vinca alkaloids, calicheamycin, mitoxantrone, cytosine arabinoside, adenosine arabinoside, fludarabine phosphate, melphalan, bleomycin, mitomycin, L-canavanine, taxoids, camptothecins, proteasome inhibitors, farnesyl-protein transferase inhibitors, epothilones, maytansinoids, discodermolide, platinum derivatives, duocarmycins, combretastatin, epipodophyllotoxins, BH3 peptides, p53 peptides, caspases, granzyme B, ribozymes, antisense oligonucleotides, c-DNAs, peptide nucleic acids, rhodamine, FITC, biotin and GFP.

52. The dual prodrug according to any of claims 49 to 51 in which the polypeptide capable of carrying the intracellularly active biologically active entity into a cell is selected from the group consisting of Antennapedia homeodomain derived peptides, Tat transactivation protein derived peptides, arginine oligomers and peptides derived from the CDR region of polyreactive anti-DNA antibodies.

53 A method of inhibiting the growth of a tumor in viva, ex viva or in vitro comprising contacting the tumor with the prodrug according to any of claims 1 to 52.

54. A method of treating neoplastic conditions comprising administering a therapeutically effective amount of a prodrug according to any of claims 1 to 52.

55. The method according to claim 53 or 54 further comprising administering a therapeutically effective amount of a second antitumor entity.

56. A pharmaceutical composition comprising the prodrug according to any of claims 1 to 52 and a pharmaceutically acceptable carrier, diluent or excipient.

57. The pharmaceutical composition according to claim 56 further comprising a second antitumor entity.

58. A method for making a prodrug according to any of claims 1 to 35 comprising the steps of:
(1) reacting a precursor of M with a precursor of L¹ under conditions in which a reactive group of M condenses with a complementary reactive group of L¹, thereby forming M−L¹; and
(2) reacting from 1 to n (M−L¹) with a precursor of B under conditions in which a reactive group of L¹ condenses with a complementary reactive group of B, thereby forming the prodrug.

59. A method for making a prodrug according to any of claims 1 to 35 comprising the steps of:
(1) reacting from 1 to n precursors of L¹ with a precursor of B under conditions in which reactive groups of (L¹)ₙ condense with complementary reactive groups of B thereby forming (L¹)ₙ−B; and
(2) reacting (L¹)ₙ−B with precursors of (M)ₙ under conditions in which reactive groups of (L¹)ₙ condense with complementary reactive groups of (M)ₙ thereby forming the prodrug.

60. A method for making a prodrug according to any of claims 36 to 52 comprising the steps of:
(1) reacting a precursor of D¹ with a precursor of L³ under conditions in which a reactive group of D¹ condenses with a complementary reactive group of L³, thereby forming D¹−L³; and
(2) reacting from 1 to n (D¹−L³) with a precursor of D² under conditions in which a reactive group of L³ condenses with a complementary reactive group of D², thereby forming the prodrug.

61. A method for making a prodrug according to any of claims 36 to 52 comprising the steps of:
(1) reacting from 1 to n precursors of L³ with a precursor of D² under conditions in which reactive groups of (L³)ₙ condense with complementary reactive groups of D² thereby forming (L³)ₙ−D²; and
(2) reacting (L³)ₙ−D² with (D¹)ₙ under conditions in which a reactive groups of (L³)ₙ condense with complementary reactive groups of (D¹)ₙ thereby forming the prodrug.
